(19) United States
(12) Patent Application Publication
(10) Pub. No.: US 2023/0256027 A1
(43) Pub. Date: Aug. 17, 2023

(54) Use of Liver Progenitor or Stem Cells, Lysates Thereof, and/or Conditioned Medium in Disorders Characterized by Vascular Hyperpermeability

(71) Applicant: CELLAÏON SA, Mont-Saint-Guibert (BE)

(72) Inventors: Etienne SOKAL, Hoeilaart (BE); Elisa CORRITORE, Mechelen (BE); Sebastien MICHEL, Bouge (BE); Sandrine HORMAN, Dion-Valmont (BE); Marine ANGE, Brussels (BE)

(21) Appl. No.: 17/766,527

(22) PCT Filed: Oct. 8, 2020
(86) PCT No.: PCT/EP2020/078226

§ 371 Date: Apr. 4, 2022

(30) Foreign Application Priority Data
Oct. 9, 2019 (EP) 19202318.2

(51) Int. Cl. A61K 35/407; C12N 5/071; A61P 7/10; A61P 9/00; A61P 13/12; A61P 11/00; A61P 31/00

(52) U.S. Cl. CPC A61K 35/407; A61P 7/10; A61P 9/00; A61P 11/00; A61P 13/12; A61P 31/00; C12N 5/0672; C12N 2500/36; C12N 2501/12; C12N 2501/15

(57) ABSTRACT

The current invention concerns liver progenitor or stem cells, lysates thereof, and/or conditioned medium obtainable by culturing liver progenitor or stem cells in said medium for use in the treatment of diseases and/or conditions caused by increased vascular permeability or for use in restoring the vascular integrity of cells and tissues in a subject following inflammation and/or infection in said subject. More particularly, the present invention relates to liver progenitor or stem cells or conditioned medium obtainable by culturing liver progenitor or stem cells in said medium for therapeutic use in sepsis and sepsis-induced diseases, such as myocardial edema, acute kidney injury and lung sepsis.

### TECHNICAL FIELD

The invention pertains to the technical field of liver progenitor or stem cells, lysates thereof, and/or conditioned medium obtainable by culturing said liver progenitor or stem cells, for therapeutic and prophylactic purposes. In particular, said therapeutic and prophylactic purposes relate to disorders and/or conditions caused by increased vascular permeability due to disturbance of the vascular integrity, e.g. following inflammation and/or infection. The current invention also pertains to activation of the AMPK pathway by means of the cells or derivatives as described above.

### BACKGROUND

The vasculature, composed of vessels of different morphology and function, distributes blood to all tissues and maintains physiological tissue homeostasis. The entire vascular circulatory system is lined by endothelial cells that form a dynamic barrier between the blood and the tissues. In resting conditions, endothelial cells, besides regulating the blood vessel tone, display very important other functions including fluid filtration, hemostasis, neutrophil recruitment, and hormone trafficking. Furthermore, a good structure of the endothelium restricts the extravasation of larger molecules and cells from blood to tissue.

Defective endothelial barrier function is a common feature of many disorders including cancer and chronic inflammatory conditions. Indeed, disintegration of the vascular barrier is closely associated to an increased leakage of larger molecules and cells which results in edema, inflammation, and often to disease progression if the leakage becomes chronic. The mechanisms underlying vascular leak could be organ-specific and depend on the specialized vasculature. Two main models have been currently proposed: while the first one will depend on the formation of trans-endothelial channels from vesicles or vacuoles, the vesiculo-vacuolar organelle (VVO), the second model involves inter-endothelial junctions (IEJs); four different types have been described so far including tight junctions, gap junctions, adherens junctions and syndesmos - that can be transiently dissolved and allow extravasation.

Vascular leak may result in excessive formation of new, unstable, and hyperpermeable vessels with poor blood flow, which further promotes hypoxia and disease propagation. Chronic vessel permeability may also facilitate metastatic spread of cancer. It is also a significant problem in vascular inflammation associated with trauma, ischemia-reperfusion injury, sepsis, adult respiratory distress syndrome, diabetes, thrombosis and cancer. An important mechanism underlying this process is increased via paracellular leakage of plasma fluid and proteins. Thus, specifically suppressing excess vascular permeability could be of a therapeutic benefit in a range of diseases as those mentioned above.

Sepsis is one of the leading causes of death in the worldwide, particularly in the developing countries, and the costs to health care systems are huge. Sepsis is the most common cause of death in the intensive care unit, and sepsis-induced disorders such as sepsis-associated acute kidney injury represent an independent and additional risk factor for mortality. Sepsis and septic shock represent a heterogeneous spectrum of complex biology and pathophysiology. Although substantial progress has been made in the understanding of fundamental mechanisms of sepsis, translation of these advances into clinically effective therapies has been disappointing. Novel therapies, outside of antibiotics, fluid resuscitation, and basic supportive care are hot topics for clinicians and investigators in this field.

Stem cells may be defined as cells capable of self-renewal and at the same time endowed with the ability to differentiate practically into all types of human cells. There are basically two main groups of stem cells - the first consists of embryonic stem cells (ESCs), which are located in the inner cell mass of the emerging blastocyst; the second group consists of somatic stem cells (SSCs) which are present in all tissues but display limited differentiation potential. Somatic stem cells include hematopoietic stem cells located in the bone marrow and representing hematopoiesis progenitors and non-hematopoietic stem cells among which the so-called mesenchymal stem cells (MSCs). Mesenchymal stem cells are currently the center of attention due to their unique properties. Allogeneic or autologous MSCs ameliorate symptoms caused by inflammation, ischemia, or physical damage to living tissues.

MSCs are pleiotropic cells able to behave differentially depending on the extracellular environment especially those containing cytokines. Besides their immunomodulatory, immunosuppressive and anti-fibrotic properties, MSCs were shown to stimulate endogenous regenerative potential of the tissues. For example, bone marrow-derived mesenchymal stem cells are known to naturally support hematopoiesis by secreting a number of trophic molecules, including soluble extracellular matrix glycoproteins, cytokines and growth factors. Studies have shown that MSCs are hypo-immunogenic, can inhibit the development of an immune response, and skew diverse immune cell populations from pro-inflammatory towards anti-inflammatory/regulatory phenotypes. In this context, the immune-reprogramming properties of cell-based therapy using MSCs represent an emerging therapeutic strategy in sepsis and associated organ dysfunction. It has been highlighted in different animal studies that MSCs may provide a promising new treatment for sepsis, but at this stage, none of these approaches has led to a new commercialized treatment.

EP 1 969 118 reports isolated liver progenitor or stem cells, also called ADHLSCs, originated from adult liver, for use in hepatology, inborn errors of liver metabolism, transplantation, infectious diseases and/or liver failure. EP 1 969 118 reports the methods to isolate and characterize these cells, and their use for transplantation or for artificial organ devices. The characterization of liver progenitor cells of EP 1 969 118 and the methods of preparing thereof are herein fully incorporated by reference. Isolation of progenitor cells or stem cells from a liver or part of a liver is performed according to methods known in the art, for example as described in EP 1 969 118, EP 3 039 123, EP 3 140 393 or EP 3 423 566.

ADHLSCs and HALPCs (human allogeneic liver progenitor cells) produced at higher scale for pursuing clinical studies, generated under GMP conditions, also referred in literature as HHALPCs) are undifferentiated progenitor cells obtained after collagenase digestion of the normal adult liver and primary culture of the isolated parenchymal fraction. These human liver progenitor or stem cells display self-renewing capacity and have the particularity to express both mesenchymal and hepatocytic markers and to display advanced hepatogenic differentiation potential. These cells are of particular interest in targeting human liver diseases, including liver fibrosis, non-alcoholic steatohepatitis (NASH) and acute-on-chronic liver failure (ACLF), and other human diseases.

WO 2015 001 124 relates to cell-free compositions obtained by culturing adult-derived human liver stem/progenitor cells in cell culture medium and isolating the resulting conditioned medium. WO 2015 001 124 describes the use of these cell free compositions for the treatment of diseases involving organ injury, organ failure as well as for the use in organ or cell transplantation, in particular within the liver.

WO 2016 200 340 describes human liver stem cells with specific markers for use in the treatment of infectious disease, chronic liver failure and liver cancer.

EP 3 016 665 relates to a cell-free conditioned medium obtainable by culturing adult derived human liver stem/progenitor cells in a cell culture medium and separating the cell culture medium from the cells.

EP 1 969 118 finally discloses human liver progenitor or stem cells with specific markers for use in the treatment of liver disease, liver cancer and liver infections.

### SUMMARY OF THE INVENTION

The present invention aims at providing a therapeutic use of liver progenitor or stem cells, lysates thereof, and/or a conditioned medium obtainable by culturing said liver progenitor or stem cells according to claim 1. Examples of useful adult liver derived progenitor or stem cells, cell lines thereof or cell populations for the current purpose are disclosed in EP 1 969 118, EP 3 039 123, EP 3 140 393 and EP 3 423 566, and are incorporated as a reference herein. Possible useful conditioned medium obtained by culturing adult-derived human liver progenitor or stem cells in cell culture medium are in detail reported in WO 2015 001 124 and herein incorporated as a reference.

The barrier function of endothelium derives from the integrity of the endothelial structure that is provided by membrane intercellular junctions. An alteration of these junctions promotes vascular hyperpermeability to fluid and solutes and can contribute significantly to damage associated with a variety of pathological states such as sepsis, cancer, organ failure and the like. The structural and functional integrity of the intercellular junctions is a major determinant of vascular permeability.

Among different roles in the cells, AMP-activated protein kinase (AMPK) is known to regulate inter-endothelial junctions (IEJs) and is thus involved in the preservation of functional integrity of endothelial cells. By modulating the endothelial barrier integrity, AMPK acts as a defense against septic injury and hyperpermeability. Accordingly, the activation of AMPK in endothelial cells provides a potential beneficial effect on the functional integrity of the vascular endothelium.

It has been unexpectedly found that liver progenitor or stem cells, the lysate obtained by the lysis of said cells and/or a conditioned medium obtainable by culturing said liver progenitor or stem cells, or part thereof, can be used in conditions connected to increased or altered vascular permeability, and/or to modulate or influence impaired vascular permeability.

Said liver progenitor or stem cells, lysates thereof and/or conditioned medium obtainable by culturing said liver progenitor or stem cells, or part thereof, facilitate the assembly or restore the formation of tight and adherens junctions through the activation of AMPK.

These effects suggest a potential beneficial mechanism of action that facilitates the restoration of the functional structure of the endothelium in inflammatory and infection conditions.

The preferred embodiments are shown as dependent claims 2-19.

The present invention also relates to a conditioned medium obtained from culturing human liver progenitor cells, according to claim 20. In particular, said medium comprises at least 30 ng/million total cells of sphingosine-1-phospate.

### DESCRIPTION OF FIGURES

The tested conditions are the same in FIGS. 1 to 5 and are listed hereafter:
The ‘control’ condition means that HDBECs are in EGM-2MV medium. EGM-2MV medium is used as control medium. In said control medium the HDBECs are not contacted with liver progenitor or stem cells of current invention, i.e. HepaStem.
The ‘HepaStem CM’ condition means that HDBECs are exposed to the conditioned medium obtained by culturing of said liver progenitor or stem cells.
The ‘Control LPS’ condition means that HDBECs are stimulated by LPS alone and are not exposed to the conditioned medium of current invention.
The ‘HepaStem CM LPS’ condition means that HDBECs are exposed to said conditioned medium and then stimulated by LPS.

FIG. 1 shows activation of AMPK after incubation of endothelial cells with conditioned medium of liver stem cells, as assessed by the analysis of ACC (acetyl-CoA carboxylase) phosphorylation. FIG. 1A visualizes a representative western blot. AICAR: 5-aminoimidazole-4-carboxamide ribonucleotide treated group. FIG. 1B visualizes the relative ACC phosphorylation of the four tested conditions.

FIG. 2 shows ZO-1 immunostaining and cell junctions’ pattern in the endothelial cells in basal state (without LPS treatment), as well as after LPS treatment, and after LPS treatment and administration of conditioned medium from liver stem cells. FIG. 2A visualizes the results obtained by using conditioned medium obtained from cultured liver progenitor or stem cells of Donor 1 (400x); FIG. 2B visualizes the results obtained by using the conditioned medium obtained from cultured liver progenitor or stem cells of Donor 2 (400x); and FIG. 2C visualizes the results obtained by using the conditioned medium obtained from cultured liver progenitor or stem cells of Donor 3 (400x).

FIG. 3 shows the effect of conditioned medium from liver stem cells in comparison to the control group on Gap junctions, tested with and without LPS treatment. The Cx43 immunostaining results (200x) are shown for the conditioned medium obtained from cultured liver progenitor or stem cells of Donor 3.

FIG. 4 shows the effect of conditioned medium from liver stem cells on adherens junctions of endothelial cells assessed by immunostaining of VE-cadherin. FIGS. 4A, 4B and 4C are results obtained by using conditioned medium obtained from culturing liver progenitor or stem cells of Donor 1, 2 and 3, respectively (200x).

FIG. 5 shows effects of conditioned medium of the invention on endothelial permeability. Each point represents a donor, i.e. results for Donor 1, 2 and 3, respectively.

FIG. 6 shows the effect of S1P3 inhibition on the prevention of LPS-induced endothelial permeability by conditioned medium of the invention.

### DEFINITIONS

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

As used herein, the term “isolated cell” refers generally to a cell that is not associated with one or more cells or one or more cellular components with which the cell is associated in vivo. For example, an isolated cell may have been removed from its native environment, or may result from propagation, e.g., ex vivo propagation, of a cell that has been removed from its native environment.

The term “in vitro” as used herein denotes outside, or external to, animal or human body. The term “in vitro” as used herein should be understood to include “ex vivo”.

The term “ex vivo” typically refers to tissues or cells removed from an animal or human body and maintained or propagated outside the body, e.g., in a culture vessel.

The term “liver progenitor cell” refers to an unspecialized and proliferation-competent cell which is produced by culturing cells that are isolated from liver or part thereof and which or the progeny of which can give rise to at least one relatively more specialized cell type. A liver progenitor cell give rise to descendants that can differentiate along one or more lineages to produce increasingly more specialized cells (but preferably hepatocytes or hepato-active cells), wherein such descendants may themselves be progenitor cells, or even to produce terminally differentiated liver cells (e.g. fully specialized cells, in particular cells presenting morphological and functional features similar to those of primary human hepatocytes).

The term “stem cell” refers to a progenitor cell capable of self-renewal, i.e., can proliferate without differentiation, whereby the progeny of a stem cell or at least part thereof substantially retains the unspecialized or relatively less specialized phenotype, the differentiation potential, and the proliferation competence of the mother stem cell. The term encompasses stem cells capable of substantially unlimited self-renewal, i.e., wherein the capacity of the progeny or part thereof for further proliferation is not substantially reduced compared to the mother cell, as well as stem cells which display limited self-renewal, i.e., wherein the capacity of the progeny or part thereof for further proliferation is demonstrably reduced compared to the mother cell.

The term “liver progenitor or stem cells” refers to adult-derived human liver stem/progenitor cells (ADHLSC) and to human allogeneic liver progenitor cells (HALPC or HALPCs) produced at higher scale for pursuing clinical studies, and is used synonymously with “human adult liver-derived progenitor cells”, “heterologous human adult liver-derived progenitor cells”, abbreviated as “HHALPC” or “HHALPCs”. These cells represent a specific type of human liver-derived progenitor cells, obtainable as described herein.

Based on the ability to give rise to diverse cell types, a progenitor or stem cell may be usually described as totipotent, pluripotent, multipotent or unipotent. A single “totipotent” cell is defined as being capable of growing, i.e. developing, into an entire organism. A “pluripotent” cell is not able of growing into an entire organism, but is capable of giving rise to cell types originating from all three germ layers, i.e., mesoderm, endoderm, and ectoderm, and may be capable of giving rise to all cell types of an organism. A “multipotent” cell is capable of giving rise to at least one cell type from each of two or more different organs or tissues of an organism, wherein the said cell types may originate from the same or from different germ layers, but is not capable of giving rise to all cell types of an organism. A “unipotent” cell is capable of differentiating to cells of only one cell lineage.

The term “mesenchymal stem cells” is to be understood as multipotent stromal cells derived or isolated from principally mesenchymal or from stromal cells. Said mesenchymal stem cells are able to differentiate into various cell types, including but not limiting to hepatocytes, osteoblasts, chondrocytes, tenocytes and adipocytes.

The term “hepatocyte” encompasses epithelial and parenchymal liver cells, including but not limited to hepatocytes of different sizes or ploidy (e.g., diploid, tetraploid, octoploid).

The term “extract” or “lysate”, as used herein, means a lysed cell content. By preference, such extract or lysate has not been further purified and thus contains the whole cell lysate content. In another preference, extract refers to a protein extract, an RNA extract, a lipid, or a membrane vesicle.

The term “fraction” as used herein, refers to a part, composition or derivative obtainable from the conditioned medium of the invention.

The term “lipopolysaccharide” or abbreviated “LPS” as used herein, means a single component of the complex pathogen associated molecular patterns, released by Gram-negative organisms. LPS are large molecules consisting of a lipid and a polysaccharide composed of O-antigen, outer core and inner core joined by a covalent bond and they are found in the outer membrane of Gram-negative bacteria.

The term “liver” refers to the liver organ. The term “part of liver” generally refers to a tissue sample derived from any part of the liver organ, without any limitation as to the quantity of the said part or the region of the liver organ where it originates. Preferably, all cell types present in the liver organ may also be represented in the said part of liver. Quantity of the part of liver may at least in part follow from practical considerations to the need to obtain enough primary liver cells for reasonably practicing the method of the invention. Hence, a part of liver may represent a percentage of the liver organ (e.g. at least 1%, 10%, 20%, 50%, 70%, 90% or more, typically w/w). In other non-limiting examples, a part of liver may be defined by weight (e.g. at least 1 g, 10 g, 100 g, 250 g, 500 g, or more).

For example, a part of liver may be a liver lobe, e.g., the right lobe or left lobe, or any segment or tissue sample comprising a large number of cells that is resected during split liver operation or in a liver biopsy.

The term “adult liver” refers to the liver of subjects that are post-natal, i.e. any time after birth, preferably full term, and may be, e.g., at least at least 1 day, 1 week, 1 month or more than 1 month of age after birth, or at least 1, 5, 10 years or more. Hence, an “adult liver”, or mature liver, may be found in human subjects who would otherwise be described in the conventional terms of “infant”, “child”, “adolescent”, or “adult”. A skilled person will appreciate that the liver may attain substantial developmental maturity in different time postnatal intervals in different animal species, and can properly construe the term “adult liver” with reference to each species.

The term “mammal” includes any animal classified as such, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes.

The term “disassociating” as used herein generally refers to partly or completely disrupting the cellular organization of a tissue or organ, i.e., partly or completely disrupting the association between cells and cellular components of a tissue or organ. As can be understood by a skilled person, the aim of disassociating a tissue or organ is to obtain a suspension of cells, i.e. a cell population, from said tissue or organ. The suspension may comprise solitary or single cells, as well as cells physically attached to form clusters or clumps of two or more cells. Disassociating preferably does not cause or causes as small as possible reduction in cell viability.

As used herein, the term “primary cell” includes cells present in a suspension of cells obtained from a tissue or organ of a subject, e.g. liver, by disassociating cells present in such explanted tissue or organ with appropriate techniques.

The term “culturing” is common in the art and broadly refers to maintenance and/or growth of cells and/or progeny thereof.

The term “passaging” is common in the art and refers to detaching and dissociating the cultured cells from the culture substrate and from each other. For sake of simplicity, the passage performed after the first time of growing the cells under adherent culture conditions is herein referred to as “first passage” (or passage 1, P1) within the method of the invention. The cells may be passaged at least one time and preferably two or more times. Each passage subsequent to passage 1 is referred to herein with a number increasing by 1, e.g., passage 2, 3, 4, 5, or P1, P2, P3, P4, P5, etc.

The term “confluence” as used herein refers to a density of cultured cells in which the cells contact one another covering substantially all of the surfaces available for growth (i.e., fully confluent).

The term “plasma” is as conventionally defined and refers to a composition which does not form part of a human or animal body.

The term “serum”, as conventionally defined, is obtained from a sample of whole blood by first allowing clotting to take place in the sample and subsequently separating the so formed clot and cellular components of the blood sample from the liquid component (serum) by an appropriate technique, typically by centrifugation. An inert catalyst, e.g., glass beads or powder, can facilitate clotting. Advantageously, serum can be prepared using serum separator tubes (SST), which contain the inert catalyst to mammals.

The term “cell medium” or “cell culture medium” or “medium” refers to an aqueous liquid or gelatinous substance comprising nutrients which can be used for maintenance or growth of cells. Cell culture medium can contain serum or can be serum-free.

The term “growth factor” as used herein refers to a biologically active substance which influences proliferation, growth, differentiation, survival and/or migration of various cell types, and may effect developmental, morphological and functional changes in an organism, either alone or when modulated by other substances. A growth factor may typically act by binding, as a ligand, to a receptor (e.g., surface or intracellular receptor) present in cells. A growth factor herein may be particularly a proteinaceous entity comprising one or more polypeptide chains. The term “growth factor” encompasses the members of the fibroblast growth factor (FGF) family, bone morphogenic protein (BMP) family, platelet derived growth factor (PDGF) family, transforming growth factor beta (TGF-β) family, nerve growth factor (NGF) family, the epidermal growth factor (EGF) family, the insulin related growth factor (IGF) family, the hepatocyte growth factor (HGF) family, the interleukin-6 (IL-6) family (e.g. oncostatin M, OSM), hematopoietic growth factors (HeGFs), the platelet-derived endothelial cell growth factor (PD-ECGF), angiopoietin, vascular endothelial growth factor (VEGF) family, or glucocorticoids. Where the method is used for human liver cells, the growth factor used in the present method may be a human or recombinant growth factor. The use of human and recombinant growth factors in the present method is preferred since such growth factors are expected to elicit a desirable effect on cellular function.

The term “cytokine” as used herein, refers to a signaling molecule such as a growth, differentiation or chemotrophic factor secreted by immune or other cells, whose action is on cells of the immune system or on multipotent cells, such as, but not limited to, T-cells, B-cells, NK cells, macrophages, tissue cells, multipotent cells including hematopoietic cells, mesenchymal stem cells and progenitor cells, or other cell types. Representative cytokines include, but are not limited to, indoleamine 2,3-dioxygenase (IDO), prostaglandin E2 (PGE2), prostaglandin-endoperoxide synthase 2 (PTGS2), hepatocyte growth factor (HGF), the group consisting of interleukins such as but not limited to interleukin-1 alpha (IL-1α), interleukin-1 beta (IL-1β), interleukin-2 (IL-2), interleukin-3 (IL-3), interleukin-4 (IL-4), interleukin-6 (IL-6), interleukin-10 (IL-10), interferons such as interferon-alpha (INF-α) and interferon-gamma (INF-γ), tumor necrosis factor-alpha (TNF-α), and granulocyte-macrophage colony stimulating factor (GM-CSF).

The terms “cell population” and “population of cells” refer generally to a group of cells. Unless indicated otherwise, the term refers to a cell group consisting essentially of or comprising cells as defined herein. A cell population may consist essentially of cells having a common phenotype or may comprise at least a fraction of cells having a common phenotype. Cells are said to have a common phenotype when they are substantially similar or identical in one or more demonstrable characteristics, including but not limited to morphological appearance, the level of expression of particular cellular components or products (e.g., RNA or proteins), activity of certain biochemical pathways, proliferation capacity and/or kinetics, differentiation potential and/or response to differentiation signals or behavior during in vitro cultivation (e.g., adherence or monolayer growth). Such demonstrable characteristics may therefore define a cell population or a fraction thereof. A cell population may be “substantially homogeneous” if a substantial majority of cells have a common phenotype. A “substantially homogeneous” cell population may comprise at least 60%, e.g., at least 70%, at least 80%, at least 90%, at least 95%, or even at least 99% of cells having a common phenotype, such as the phenotype specifically referred to (e.g., the phenotype of liver progenitor or stem cells of the invention, or to progeny of liver progenitor or stem cells of the invention). Moreover, a cell population may consist essentially of cells having a common phenotype such as the phenotype of liver progenitor or stem cells of the invention (i.e. a progeny of liver progenitor or stem cells of the invention) if any other cells present in the population do not alter or have a material effect on the overall properties of the cell population.

The abbreviation “AMPK” as used herein refers to 5′ AMP-activated protein kinase or 5′ adenosine monophosphate-activated protein kinase, an enzyme that plays a role in cellular energy homeostasis, largely to activate glucose and fatty acid uptake and oxidation when cellular energy is low.

The term “pharmaceutically acceptable carrier” as used herein, refers to a carrier or a diluent that does not cause significant irritation to a subject and does not abrogate the biological activity and properties of the administered composition. Examples, without limitations, of carriers are propylene glycol, saline, emulsions and mixtures of organic solvents with water.

The term “disorder”, as used herein, means disruption of the systematic function and physiological structure of the organ/system of organs.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The term “allogeneic” as used herein means that the donated material comes from different, although often related, individual than the recipient. Allogeneic stem cell transplantation refers to a procedure in which a person receives stem cells from a genetically similar, but not identical, donor. This is often a sister or brother but could be an unrelated donor.

The term “vascular permeability” as used herein is understood as the capacity of a blood vessel wall to allow for the flow of small molecules (drugs, nutrients, water, ions) or even whole cells in and out of the vessel.

The term “increased vascular permeability”, as used herein, implies an increased passage of various molecules through the blood vessel wall, including passage of those molecules which may be the cause of acute and chronic inflammation, cancer, and wound healing. Said increased permeability or even hyperpermeability may be mediated by acute or chronic exposure to vascular permeabilizing agents, particularly vascular permeability factor/vascular endothelial growth factor (VPF/VEGF, VEGF-A).

The term “vascular integrity” as used herein, refers to proper functions of various components of the blood vessel wall which maintain vascular homeostasis. One of the early hallmarks of deteriorating vascular integrity is increased permeability which is predominantly controlled by endothelial junction stability. Selective regulation of vascular permeability is achieved by regulation of the size and state of paracellular gaps and control of the transcellular transport.

The term “heart disease” as used herein, refers to any disorder and/or condition that affects the heart. Said heart diseases may include blood vessel diseases, such as coronary artery disease; heart rhythm problems (arrhythmias); and congenital heart defects, among others.

The term “heart disease” is often used interchangeably with the term “cardiovascular disease”. Cardiovascular disease generally refers to conditions that involve narrowed or blocked blood vessels that can lead to a heart attack, chest pain (angina) or stroke. Other heart conditions, such as those that affect the heart’s muscle, valves or rhythm, also are considered forms of heart disease.

The term “pulmonary disease” as used herein, refers to any lung disease which is any problem in the lungs that prevents the lungs from working properly.

The term “ischemic disease” as used herein refers any disease and/or condition which is connected to impaired tissue/organ or organ system function, due to decrease of a blood flow and sufficient supplementation of said tissue/organ or organ system with oxygen. The types of ischemic disease include, but are not limited to, coronary heart disease, cerebral or brain ischemia, pulmonary ischemia, renal ischemia and the like.

The term “diabetes” as used herein, refers to a group of metabolic disorders in which there are high blood sugar levels over a prolonged period. Symptoms of high blood sugar include frequent urination, increased thirst, and increased hunger. Diabetes can cause many complications, especially if left untreated. Acute complications can include diabetic ketoacidosis, hyperosmolar hyperglycemic state, or death. Serious long-term complications include cardiovascular disease, stroke, chronic kidney disease, foot ulcers, and damage to the eyes.

The term “ocular disease” as used herein, refers to disorders and conditions affecting an eye.

The term “cancer” as used herein, refers to a group of diseases involving abnormal cell growth with the potential to invade or spread to other parts of the body.

The term “solid tumor” as used herein, refers to an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias generally do not form solid tumors.

The term “Clarkson’s disease” as used herein, refers to an idiopathic systemic capillary leak syndrome (SCLS), a rare disorder characterized by transient episodes of hypotensive shock and anasarca thought to arise from reversible microvascular barrier dysfunction. This potentially fatal disorder is characterized by stereotypic ‘attacks’ of varying intensity of hypovolemic shock and generalized edema in association with hemoconcentration (as detected by an elevated hematocrit concentration) and hypoalbuminemia, typically occurring in the absence of albuminuria.

The term “sepsis” as used herein means a life-threatening organ dysfunction caused by a dysregulated host response to infection. Sepsis is characterized by disseminated inflammatory response elicited by microbial infections. Sepsis commonly leads to a state of immunosuppression characterized by lymphocyte apoptosis and susceptibility to nosocomial infections.

The term “sepsis-induced” as used herein, refers to all diseases and conditions that occur in a tissue, organ and/or organ system, which are the consequence of sepsis and related events. Such conditions may include, but are not limited to sepsis-induced cardiomyopathy, sepsis-induced coagulopathy, poor organ function, acute kidney injury, lung sepsis, poor organ function and/or insufficient blood flow, and the like.

The term “septic cardiomyopathy” or “sepsis-induced myocardial edema” as used herein, means a cardiovascular complication in patients with severe sepsis, characterized by a reversible decrease in systolic and/or diastolic left ventricular (LV) function, associated with left ventricular wall edema during sepsis.

The term “cecal ligation and puncture” refers to a disorder which consists on the perforation of the cecum allowing the release of fecal material into the peritoneal cavity to generate an exacerbated immune response induced by polymicrobial infection. The polymicrobial sepsis is generated from fecal spillage after needle puncture. Cecal ligation and puncture mode, represents a gold standard and the most frequently used model to study sepsis, because it closely resembles the progression and characteristics of human sepsis.

The term “sepsis induced lung injury” as used herein, means acute lung injury or disorder, which occurs secondary to sepsis. The lung is the organ most often affected by the sepsis related systemic inflammatory response, resulting in acute lung injury or the more severe acute respiratory distress syndrome.

### DETAILED DESCRIPTION OF THE INVENTION

The present invention concerns the use of isolated liver progenitor or stem cells, lysate thereof, and/or conditioned medium obtainable by culturing said liver progenitor or stem cells in medium, for the treatment of diseases and conditions caused by increased vascular permeability. In a further or other embodiment, said cells, lysate thereof and/or conditioned medium can be used for restoring the vascular integrity of cells and tissues in a subject following inflammation and/or infection in said subject. Alternatively or additionally, said cells, lysate thereof and/or conditioned medium can be used to modulate AMP signaling in a subject, preferably a human subject, by AMPK activation.

AMP-activated protein kinase (AMPK) is an energy sensor that is “switched on” by cellular stressors, such as hypoxia, heat-shock, ischemia, and glucose deprivation. Its activation inhibits anabolic (ATP consuming) pathways and induces stimulation of catabolic (ATP generating) pathways. AMPK, known to regulate IEJs, preserves the functional integrity of endothelial cells, endothelial barrier integrity and thus acts as defense against septic injury and hyperpermeability. Activation of AMPK in cells provides a potential beneficial effect on the functional integrity of the vascular endothelium.

Liver progenitor or stem cells and methods for isolating the latter are known in the art, evidenced for instance by EP 1 969 118, EP 3 039 123, EP 3 140 393 or EP 3 423 566, which are incorporated as references herein. The use of liver progenitor or stem cells for treating liver diseases has been equally discussed. Cell-free compositions obtained by culturing adult-derived human liver progenitor or stem cells in cell culture medium and their use for the treatment of diseases involving organ injury, organ failure as well as for use in organ or cell transplantation, in particular for liver, are the object of WO 2015 001 124.

Herein it is shown for the first time that liver progenitor or stem cells, as well as lysates thereof and conditioned medium obtained by culturing said cells in medium, are useful for the recovery of the vascular integrity of cells and tissues by facilitating the restoration of the functional structure of the endothelium, in particular via AMPK activation. As a consequence, liver progenitor or stem cells, as well as lysates thereof and conditioned medium obtained by culturing said cells in medium provide treatment for conditions and diseases which are linked to an increased vascular hyperpermeability.

The cells used in current invention are liver progenitor or stem cells, by preference mammalian cells, such as human liver progenitor or stem cells.

In one embodiment, human liver progenitor or stem cells are positive for at least one mesenchymal marker. Mesenchymal markers include but are not limited to Vimentin, CD13, CD90, CD73, CD44, CD29, a-smooth muscle actin (ASMA) and CD140b. In addition, the liver progenitor cells may secrete HGF and/or PGE2. Moreover, they can optionally be positive for at least one hepatic marker and/or exhibit at least one liver-specific activity. For example, hepatic markers include but are not limited to HNF-3B, HNF-4, CYP1A2, CYP2C9, CYP2E1, CYP3A4 and alpha-1 antitrypsin and may also include albumin (ALB). Liver-specific activities may include but are not limited to urea secretion, bilirubin conjugation, alpha-1-antitrypsin secretion and CYP3A4 activity.

In another embodiment of the present invention, human liver progenitor or stem cells express at least one mesenchymal marker selected from CD90, CD44, CD73, CD13, CD140b, CD29, vimentin and a-smooth muscle actin (ASMA), and they also secrete HGF.

In another embodiment of the present invention, human liver progenitor or stem cells express at least one mesenchymal marker selected from CD90, CD44, CD73, CD13, CD140b, CD29, vimentin and a-smooth muscle actin (ASMA), and they also secrete HGF and PGE2.

In another embodiment of the present invention, human liver progenitor or stem cells express at least one mesenchymal marker selected from CD90, CD44, CD73, CD13, CD140b, CD29, vimentin and a-smooth muscle actin (ASMA), and they optionally also express at least one hepatic marker and/or exhibit a liver-specific activity and/or exhibit a liver-specific activity.

In one embodiment, human liver progenitor or stem cells may be characterized in that they co-expresses (i.e., are positive for) at least one mesenchymal marker including but not limited to CD90, CD44, CD73, CD13, CD140b, Vimentin, CD29, and a-smooth muscle actin (ASMA), with at least one hepatic or hepatocyte marker including but not limited to alpha-fetoprotein (AFP), alpha-1 antitrypsin, HNF-4 and/or MRP2 transporter, optionally with the hepatocyte marker albumin (ALB). They optionally also exhibit a liver-specific activity which may be selected from urea secretion, bilirubin conjugation, alpha-1-antitrypsin secretion and CYP3A4 activity. Moreover, the HALPCs preferably express HGF and PGE-2.

In one embodiment, said cells are preferably human liver progenitor or stem cells positive for at least one hepatic marker and at least one mesenchymal marker and that display at least one liver-specific activity. For example, hepatic markers include but are not limited to HNF-3B, HNF-4, CYP1A2, CYP2C9, CYP2E1, CYP3A4 and alpha-1 antitrypsin and may also include albumin (ALB). Mesenchymal markers include but are not limited to Vimentin, CD13, CD90, CD73, CD44, CD29, a-smooth muscle actin (ASMA) and CD140b. Liver-specific activities include but are not limited to urea secretion, bilirubin conjugation, alpha-1 antitrypsin secretion and CYP3A4 activity.

Said liver progenitor or stem cell, or a cell population comprising such cells will be able to give rise to at least hepatocyte like cells. By preference, said cells do not differentiate into osteocytes or adipocytes.

In a preferred embodiment of the current invention, the used human liver progenitor or stem cells will be positive for at least one of the markers chosen from the group of a-smooth muscle actin (ASMA), albumin (ALB), CD140b, and MMP1; and negative for at least one of the markers chosen from the group sushi domain containing protein 2 (SUSD2) and cytokeratin-19 (CK-19).

In another embodiment of the present invention, the human liver progenitor or stem cells are measured positive for a-smooth muscle actin (ASMA), CD140b, and optionally albumin (ALB); and negative for cytokeratin-19 (CK-19).

In a further embodiment, the human liver progenitor or stem cells are measured positive for a-smooth muscle actin (ASMA), CD140b, and optionally albumin (ALB) and Sushi domain containing protein 2 (SUSD2); and negative for Cytokeratin-19 (CK-19).

In another embodiment of the present invention, the human liver progenitor or stem cells are measured positive for a-smooth muscle actin (ASMA), CD140b, and optionally albumin (ALB); and negative for sushi domain containing protein 2 (SUSD2) and cytokeratin-19 (CK-19).

In another embodiment of the invention, the human liver progenitor or stem cells are measured positive for CD90, CD73, vimentin and ASMA.

In a further embodiment of the invention, the human liver progenitor or stem cells are positive for CD90, CD73, vimentin and ASMA, and exhibit in average less than about 2.5% clonal aberrations per metaphase and/or less than about 15% non-clonal aberrations per metaphase.

In another embodiment, the human liver progenitor or stem cells are measured positive for CD90, CD73, vimentin and ASMA, and negative for CK-19.

In another or further preferred embodiment, said human liver progenitor or stem cells are measured positive for one or more markers chosen from the group of:
a-smooth muscle actin (ASMA), albumin (ALB), CD140b, MMP1;
at least one hepatic marker selected from HNF-3B, HNF-4, CYP1A2, CYP2C9, CYP2E1, CYP3A4;
at least one mesenchymal marker selected from vimentin, CD90, CD73, CD44, CD29;
at least one liver-specific activity selected from urea secretion, bilirubin conjugation, alpha-1 antitrypsin secretion and CYP3A4 activity;
at least one marker selected from ATP2B4, ITGA3, TFRC, SLC3A2, CD59, ITGB5, CD151, ICAM1, ANPEP, CD46, CD81; and
at least one marker selected from ITGA11, FMOD, KCND2, CCL11, ASPN, KCNK2, and HMCN1.

In still another or further embodiment of the present invention, the HALPC cell is further measured positive for:
at least one hepatic marker selected from HNF-3B, HNF-4, CYP1A2, CYP2C9, CYP2E1 and CYP3A4 and optionally albumin;
at least one mesenchymal marker selected from Vimentin, CD90, CD73, CD44, and CD29;
at least one liver-specific activity selected from urea secretion, bilirubin conjugation, alpha-1-antitrypsin secretion, and CYP3A4 activity;
at least one marker selected from ATP2B4, ITGA3, TFRC, SLC3A2, CD59, ITGB5, CD151, ICAM1, ANPEP, CD46, and CD81; and
at least one marker selected from MMP1, ITGA11, FMOD, KCND2, CCL11, ASPN, KCNK2, and HMCN1.

It will be understood that the cells can be positive for any combination of markers as given above. In a particularly preferred embodiment, said cells are positive for all markers above.

In another or further embodiment, said cells are measured negative for one or more markers chosen from the group of:
sushi domain containing protein 2 (SUSD2) and cytokeratin-19 (CK-19);
CD271;
at least one marker selected from ITGAM, ITGAX, IL1R2, CDH5, and NCAM1; and
at least one marker selected from HP, CP, RBP4, APOB, LBP, ORM1, CD24, CPM, and APOC1.

It will be understood that the cells can be negative for any combination of markers as given above. In a particularly preferred embodiment, said cells are negative for all markers above.

In a further embodiment, said cells are negative for HLA-DR.

In a further embodiment, the HALPCs are negative for certain markers, such as CD133, CD45, CK19 and/or CD31.

In a further embodiment, the HALPCs may also be measured positive for one or more of the enzymatic activities listed in WO2016/030525, Table 6. In some embodiments, this type of adult liver progenitor cell can be further characterized by a series of negative markers, in particular for one or more of the group consisting of ITGAM, ITGAX, IL1R2, CDH5, and NCAM 1. Additionally, HALPCs may also be measured negative for one or more of the group consisting of HP, CP, RBP4, APOB, LBP, ORM 1, CD24, CPM, and APOC1.

The biological activities, the markers, and the morphological/functional features listed above can be present in HALPCs in different combinations of markers, such as:
positive for a-smooth muscle actin, vimentin, CD90, CD73, CD44, CD29, CD140b, and CYP3A4 activity and optionally albumin; and
negative for Sushi domain containing protein 2, Cytokeratin-19, and CD271.

Further features can be also determined for HALPCs of the above embodiment in any functional and technical combination, for instance by measuring positivity for at least one further marker selected from ATP2B4, ITGA3, TFRC, SLC3A2, CD59, ITGB5, CD151, ICAM1, ANPEP, CD46, and CD81. In some of such embodiments, HALPCs can be measured negative for at least one further marker selected from the group consisting of ITGAM, ITGAX, IL1R2, CDH5, and NCAM1. In some of such embodiments, HALPCs can be measured negative for at least one of HP, CP, RBP4, APOB, LBP, ORM1, CD24, CPM, and APOC1.

Among the technologies used for identifying such markers and measuring them as being positive or negative, western blot, flow cytometry, immunocytochemistry, and ELISA are preferred since these allow marker detection at the protein level even with the low amount of liver progenitor or stem cells that are available at this step.

In a further preferred embodiment said liver progenitor or stem cells have mesenchymal-like morphology, involving any or all of growth in monolayers, flattened form, broad cytoplasm and ovoid nuclei with one or two nucleoli.

These progenitor or stem cells are able to retain their proliferation capacity and incubation in specific media, which allows the cells to differentiate specifically into liver-specific cell types and not into mesodermal cell types.

In an embodiment of the invention, a cell lysate of the cells as described above is used, rather than the actual cells. Said cell lysate may be obtained by any means known in the art, such as enzyme digestion of cells or a cell culture, detergent and/or buffer treatment, and the like.

In a further preferred embodiment, said liver progenitor or stem cell is human. In a particularly preferred embodiment, said human liver progenitor or stem cell is adult.

The human liver progenitor or stem cells as described above may be obtained by any suited method known in the art, for instance as described in for example as described in EP 1 969 118, EP 3 039 123, EP 3 140 393 or EP 3 423 566 (see Example 1). Briefly, a population of liver primary cells is first obtained from disassociating of liver or part thereof, to form a population of liver primary cells from said liver or part thereof. Subsequently, cells comprised in this preparation are cultured under adherent conditions, preferably as to allow adherence and growth of cells onto a support. Next, these cells are passaged at least once, preferably at 70%, 80% or 90% confluence. Finally, cells are isolated and are positive for at least one hepatic marker and at least one mesenchymal marker and that have at least one liver-specific activity.

In a preferred embodiment, such method comprises the steps of:
(a) Disassociating adult liver or a part thereof to form a population of primary liver cells;
(b) Generating preparations of the primary liver cells of (a);
(c) Culturing the cells comprised in the preparations of (b) onto a support which allows adherence and growth of cells thereto and the emergence of a population of cells;
(d) Passaging the cells of (c) at least once;
(e) Isolating the cell population that is obtained after passaging of (d) and positive for the markers identified in the Summary of the Invention.

A suitable method for disassociating liver or part thereof to obtain a population (suspension) of primary cells therefrom may be any method well known in the art, including but not limited to, enzymatic digestion, mechanical separation, filtration, centrifugation and combinations thereof.

Concerning step (a) of the method, the dissociation step involves obtaining a liver or a part thereof that comprises, together with fully differentiated hepatocytes, an amount of primary cells that can be used for producing liver progenitor or stem cells.

The liver or part thereof is obtained from a “subject”, “donor subject” or “donor”, interchangeably referring to a vertebrate animal, preferably a mammal, more preferably a human. A part of a liver can be a tissue sample derived from any part of the liver and may comprise different cell types present in the liver. The cells according to the invention are preferably isolated from mammalian liver or part of a liver, where the term mammalian refers to any animal classified as a mammal, including humans, domestic and farm animals, and zoo, laboratory, sports, or pet animals, such as dogs, horses, cats, cows, mice, rats, rabbits, etc. More preferably, the liver progenitor cell or stem cell is isolated from human liver or a part thereof, preferably human adult liver or a part thereof. Liver progenitor or stem cells, cell lines, or progeny thereof, derived according to the invention from livers of adult human subjects, can be advantageously used, e.g., in research and in therapy of patients, especially human patients, suffering from disorders involving unwanted immune responses, for example, but not limited to inflammation, auto-immune disease and graft rejection, including liver diseases. In contrast to other sources of stem cells, such as for example embryonic stem cells which are prone to generate tumor growth, stem cells derived from adult livers offer a reduction in the risk for carcinogenic deviation making them safer for use in cell transplantation.

In an alternative embodiment of the invention, the adult liver or part thereof may be from a non-human animal subject, preferably a non-human mammal subject. Progenitor or stem cells or cell lines, or progeny thereof, derived as described herein from liver of non-human animal or non-human mammal subjects can be advantageously used, e.g., in research and in the therapy of liver disease in members of the same, related or other non-human animal or non-human mammal species, or even in the therapy of human patients suffering from liver disease (e.g., xenotransplantation, bio-artificial liver devices comprising non-human animal or non-human mammal cells). By means of example and not limitation, particularly suitable non-human mammal cells for use in human therapy may originate from pigs.

A donor subject may be living or dead, as determined by art-accepted criteria, such as, for example, the “heart-lung” criteria (usually involving an irreversible cessation of circulatory and respiratory functions) or the “brain death” criteria (usually involving an irreversible cessation of all functions of the entire brain, including the brainstem). Harvesting may involve procedures known in the art, such as, for example, biopsy, resection or excision.

A skilled person will appreciate that at least some aspects of harvesting liver or part thereof from donor subjects may be subject to respective legal and ethical norms. By means of example and not limitation, harvesting of liver tissue from a living human donor may need to be compatible with sustenance of further life of the donor. Accordingly, only a part of liver may typically be removed from a living human donor, e.g., using biopsy or resection, such that an adequate level of physiological liver functions is maintained in the donor. On the other hand, harvesting of liver or part thereof from a non-human animal may, but need not be compatible with further survival of the non-human animal. For example, the non-human animal may be humanely culled after harvesting of the tissue. These and analogous considerations will be apparent to a skilled person and reflect legal and ethical standards.

Liver or part thereof may be obtained from a donor, preferably a human donor, who has sustained circulation, e.g., a beating heart, and sustained respiratory functions, e.g., breathing lungs or artificial ventilation. Subject to ethical and legal norms, the donor may need to be or need not be brain dead (e.g., removal of entire liver or portion thereof, which would not be compatible with further survival of a human donor, may be allowed in brain dead human beings). Harvesting of liver or part thereof from such donors is advantageous, since the tissue does not suffer substantial anoxia (lack of oxygenation), which usually results from ischemia (cessation of circulation).

Alternatively, liver or part thereof may be obtained from a donor, preferably a human donor, who at the time of harvesting the tissue has ceased circulation, e.g., has a non-beating heart, and/or has ceased respiratory functions, e.g., has non-breathing lungs and no artificial ventilation. While liver or part thereof from these donors may have suffered at least some degree of anoxia, viable progenitor or stem cells can also be isolated from such tissues. Liver or part thereof may be harvested within about 24 h after the donor’s circulation (e.g., heart-beat) ceased, e.g., within about 20 h, e.g., within about 16 h, more preferably within about 12 h, e.g., within about 8 h, even more preferably within about 6 h, e.g., within about 5 h, within about 4 h or within about 3 h, yet more preferably within about 2 h, and most preferably within about 1 h, such as, within about 45, 30, or 15 minutes after the donor’s circulation (e.g., heart-beat) ceased.

The harvested tissues may be cooled to about room temperature, or to a temperature lower than room temperature, but usually freezing of the tissue or parts thereof is avoided, especially where such freezing would result in nucleation or ice crystal growth. For example, the tissue may be kept at any temperature between about 1° C. and room temperature, between about 2° C. and room temperature, between about 3° C. and room temperature or between about 4° C. and room temperature, and may be advantageously be kept at about 4° C. The tissue may also be kept “on ice” as known in the art. The tissue may be cooled for all or part of the ischemic time, i.e., the time after cessation of circulation in the donor. That is, the tissue can be subjected to warm ischemia, cold ischemia, or a combination of warm and cold ischemia. The harvested tissue may be so kept for, e.g., up to 48 h before processing, preferably for less than 24 h, e.g., less than 16 h, more preferably for less than 12 h, e.g., less than 10 h, less than 6 h, less than 3 h, less than 2 h or less than 1 h.

The harvested tissue may advantageously be but need not be kept in, e.g., completely or at least partly submerged in, a suitable medium and/or may be but need not be perfused with the suitable medium, before further processing of the tissue. A skilled person is able to select a suitable medium which can support the survival of the cells of the tissue during the period before processing.

Isolation of progenitor cells or stem cells from a liver or part of a liver is performed according to methods known in the art, for example as described in EP 1 969 118, EP 3 039 123, EP 3 140 393 or EP 3 423 566 (see Example 1).

Briefly, a population of liver primary cells is first obtained from disassociating of liver or part thereof, to form a population of primary cells from said liver or part thereof. Subsequently, cells comprised in this preparation are cultured under adherent conditions, preferably as to allow adherence and growth of cells onto a support. Next, these cells are passaged at least once, preferably at 70% confluence. Finally, cells, which are positive for at least one hepatic marker and at least one mesenchymal marker and that have at least one liver-specific activity, are isolated.

Concerning step (b) of the method, the population of primary cells as defined and obtained herein by disassociating liver or part thereof may typically be heterogeneous, i.e., it may comprise cells belonging to one or more cell types belonging to any liver-constituting cell type, including progenitor or stem cells, that may have been present in liver parenchyma and/or in the liver non-parenchymal fraction. Exemplary liver-constituting cell types include but are not limited to hepatocytes, cholangiocytes (bile duct cells), Kupffer cells, hepatic stellate cells (Ito cells), oval cells and liver endothelial cells. The above terms have art-established meanings and are construed broadly herein as encompassing any cell type classified as such.

A primary cell population may comprise hepatocytes in different proportions (0.1%, 1%, 10%, or more of total cells), according to the method of disassociating liver and/or any methods for fractioning or enriching the initial preparation for hepatocytes and/or other cell types on the basis of physical properties (dimension, morphology), viability, cell culture conditions, or cell surface marker expression by applying any suitable techniques.

The population of primary cells as defined and obtained herein by disassociating liver (or part of it) can be used immediately for establishing cell cultures as fresh primary liver cells or, preferably, stored as cryopreserved preparations of primary liver cells using common technologies for their long-term preservation.

Concerning step (c), the preparation of liver primary cells obtained in step (b) is then cultured directly onto a fully synthetic support (e.g., plastic or any polymeric substance) or a synthetic support pre-coated with feeder cells, protein extracts, or any other material of biological origin that allow the adherence and the proliferation of similar primary cells and the emergence of a population of adult liver progenitor or stem cells having the desired markers, such markers being identified preferably at the level of protein, by means of immunohistochemistry, flow cytometry, or other antibody based technique. Primary cells are cultured in a cell culture medium sustaining their adherence and the proliferation of and the emergence of a homogenous cell population. This step of culturing of primary liver cells as defined above leads to emergence and proliferation of liver progenitor or stem cells in the culture and can be continued until liver progenitor or stem cells have proliferated sufficiently. For example, culturing can be continued until the cell population has achieved a certain degree of confluence (e.g., at least 50%, 70%, 80% or at least 90% or more confluent).

Liver progenitor or stem cells obtained at step (c) can be further characterized by technologies that allow detecting relevant markers already at this stage (that is, before passaging cells as indicated in step (d)), as described in EP 3 140 393 or EP 3 423 566. Among the technologies used for identifying such markers and measuring them as being positive or negative, western blot, flow cytometry, immunocytochemistry, and ELISA are preferred since these allow marker detection at the protein level even with the low amount of liver progenitor or stem cells that are available at this step.

The isolation of a liver progenitor or stem cell can then be made based on the presence of positive markers, the liver progenitor or stem cells are positive for at least one mesenchymal marker. Mesenchymal markers include but are not limited to Vimentin, CD13, CD90, CD73, CD44, CD29, a-smooth muscle actin (ASMA) and CD140-b. In addition, the liver progenitor cells may secrete HGF and/or PGE2. Moreover, they can optionally be positive for at least one hepatic marker and/or exhibit at least one liver-specific activity. For example, hepatic markers include but are not limited to HNF-3B, HNF-4, CYP1A2, CYP2C9, CYP2E1, CYP3A4 and alpha-1 antitrypsin and may also include albumin (ALB). Liver-specific activities may include but are not limited to urea secretion, bilirubin conjugation, alpha-1-antitrypsin secretion and CYP3A4 activity.

In a particular embodiment, the isolation of a liver progenitor or stem cell can be made based on the presence of positive mesenchymal marker selected from Vimentin, CD13, CD90, CD73, CD44, CD29, a-smooth muscle actin (ASMA) and CD140-b, and optionally on the secretion of HGF and/or PGE2.

In an embodiment, the isolation of a liver progenitor or stem cell can then be made based on the presence of positive markers, the liver progenitor or stem cells are positive for at least one hepatic marker and at least one mesenchymal marker and that have at least one liver-specific activity. For example, hepatic markers include but are not limited to albumin (ALB), HNF-3B, HNF-4, CYP1A2, CYP2C9, CYP2E1, CYP3A4 and alpha-1 antitrypsin. Mesenchymal markers include but are not limited to Vimentin, CD13, CD90, CD73, CD44, CD29, a-smooth muscle actin (ASMA) and CD140b. Liver-specific activities include but are not limited to urea secretion, bilirubin conjugation, alpha-1 antitrypsin secretion and CYP3A4 activity.

Concerning step (d) of the method, primary cells are cultured in a cell culture medium sustaining their adherence and the proliferation of and the emergence of a homogenous cell population that, following at least one passage, is progressively enriched for liver progenitor or stem cells. These liver progenitor or stem cells can be rapidly expanded for generating sufficient cells for obtaining progeny having the desired properties (as described in EP 3 140 393 or EP 3 423 566), with cell doubling that can be obtained within 48-72 hours and maintenance of liver progenitor or stem cells having the desired properties for at least for 2, 3, 4, 5 or more passages.

The isolated liver progenitor or stem cells are plated onto a substrate which allows adherence of cells thereto, and cultured in a medium sustaining their further proliferation, generally a liquid culture medium, which may contain serum or may be serum-free. In general, a substrate which allows adherence of cells thereto may be any substantially hydrophilic substrate. Current standard practice for growing adherent cells may involve the use of defined chemical media with or without addition of bovine, human or other animal serum. These media, that can be supplemented with appropriate mixture of organic or inorganic compounds may, besides providing nutrients and/or growth promoters, also promote the growth/adherence or the elimination/detachment of specific cell types. The added serum, besides providing nutrients and/or growth promoters, may also promote cell adhesion by coating the treated plastic surfaces with a layer of matrix to which cells can better adhere. As appreciated by those skilled in the art, the cells may be counted in order to facilitate subsequent plating of the cells at a desired density.

The environment in which the cells are plated may comprise at least a cell medium, in the methods of the invention typically a liquid medium, which supports the survival and/or growth of the isolated liver progenitor cells. The liquid culture medium may be added to the system before, together with or after the introduction of the cells thereto.

Typically, the medium will comprise a basal medium formulation as known in the art. Many basal media formulations can be used to culture the primary cells herein, including but not limited to Eagle’s Minimum Essential Medium (MEM), Dulbecco’s Modified Eagle’s Medium (DMEM), alpha modified Minimum Essential Medium (alpha-MEM), Basal Medium Essential (BME), Iscove’s Modified Dulbecco’s Medium (IMDM), BGJb medium, F-12 Nutrient Mixture (Ham), Liebovitz L-15, DMEM/F-12, Essential Modified Eagle’s Medium (EMEM), RPMI-1640, Medium 199, Waymouth’s MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as Williams Medium E, IMDM or DMEM, which are reported to sustain in vitro culture of adult liver cells, and including a mixture of growth factors for their appropriate growth, proliferation, maintenance of desired markers and/or biological activity, or long-term storage. Another preferred medium is commercially available serum-free medium that supports the growth of liver progenitor or stem cells, such as e.g. StemMacs™ from Miltenyi, Prime-XV from FUJIFILM Irvine Scientific or DMEM 10 % FBS (Gibco).

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

For use in culture, basal media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks’ Balanced Salt Solution (HBSS), Earle’s Salt Solution. Further antioxidant supplements may be added, e.g., β-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be advantageously used in cell culture and include, but are not limited to D-aldosterone, diethylstilbestrol (DES), dexamethasone, estradiol, hydrocortisone, insulin, prolactin, progesterone, somatostatin/human growth hormone (HGH), thyrotropin, thyroxine, L-thyronine, epithelial growth factor (EGF) and hepatocyte growth factor (HGF). Liver cells can also benefit from culturing with triiodothyronine, a-tocopherol acetate, and glucagon.

Lipids and lipid carriers can also be used to supplement cell culture media. Such lipids and carriers can include, but are not limited to cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulations.

Also contemplated is supplementation of cell culture medium with mammalian plasma or serum. Plasma or serum often contains cellular factors and components that are necessary for viability and expansion. The use of suitable serum replacements is also contemplated. Suitable plasma or serum for use in the media as described herein may include human plasma or serum; or plasma or serum derived from non-human animals, preferably non-human mammals, such as, e.g., non-human primates (e.g., lemurs, monkeys, apes), fetal or adult bovine, horse, porcine, lamb, goat, dog, rabbit, mouse or rat serum or plasma, etc. In another embodiment, the any combination of the above plasma and/or serum may be used in the cell medium.

When passaged, the cultured cells are detached and dissociated from the culture substrate and from each other. Detachment and dissociation of the cells can be carried out as generally known in the art, e.g., by enzymatic treatment with proteolytic enzymes (e.g., chosen from trypsin, collagenase, e.g., type I, II, III or IV, dispase, pronase, papain, etc.), treatment with bivalent ion chelators (e.g., EDTA or EGTA) or mechanical treatment (e.g., repeated pipetting through a small bore pipette or pipette tip), or any combination of these treatments.

A suitable method of cell detachment and dispersion should ensure a desired degree of cell detachment and dispersion, while preserving a majority of cells in the culture. Preferably, the detachment and dissociation of the cultured cells would yield a substantial proportion of cells as single, viable cells (e.g., at least 50%, 70%, 80%, 90% of the cells or more). The remaining cells may be present in cell clusters, each containing a relatively small number of cells (e.g., on average, between 1 and 100 cells).

Next, the so detached and dissociated cells (typically as a cell suspension in an isotonic buffer or a medium) may be re-plated onto a substrate which allows the adherence of cells thereto, and are subsequently cultured in a medium as described above sustaining the further proliferation of HALPCs and HALPC progeny. These cells may be then cultured by re-plating them at a density of between 10 and 10⁵ cells/cm², and at a splitting ratio between about 1/16 and ½, preferably between about ⅛ and ½, more preferably between about ¼ and ½. The splitting ratio denotes the fraction of the passaged cells that is seeded into an empty (typically a new) culture vessel of the same surface area as the vessel from which the cells were obtained. The type of culture vessel, as well as of surface allowing cell adherence into the culture vessel and the cell culture media, can be the same as initially used and as described above, or may be different. Preferably, cells are maintained onto CellBind or any other appropriate support that is coated with extracellular matrix proteins (such as collagens, and preferably collagen type I) or synthetic peptides that are acceptable in GMP conditions.

Concerning step (e) above, the isolation of population of HALPCs applies to cells that are positive for the listed markers, further validating the criteria for initially identifying HALPCs at step (c) above but that can be more easily established given the higher amount of cells that are available after passaging.

The current invention equally relates to a conditioned medium obtainable by culturing human liver progenitor or stem cells for uses as described above. In addition to the cells as such, also the medium that was harvested from the cell cultures, commonly known as conditioned medium, was shown to facilitate the assembly of tight and adherens junctions of endothelial cells.

In a particularly preferred embodiment, said conditioned medium of the invention is cell-free. The cell-free nature can be obtained by conventional methods in the art such as filtration, enzymatic digestion, centrifugation, absorption, and/or separation by chromatography, or repetitions and/or combinations of such methods. Said conditioned medium further comprises soluble proteins, microvesicles and exosomes.

The conditioned medium obtainable by culturing the liver progenitor or stem cells described above was found to comprise soluble proteins, among others, growth factors, chemokines, matrix metalloproteases, and pro- and anti-inflammatory cytokines whose presence can provide useful biological activities. These components are presumed to be secreted by the cells in the medium.

In one embodiment, the isolated liver progenitor or stem cells are plated onto a substrate which allows adherence of cells thereto, and cultured in a medium sustaining their further proliferation, generally a liquid culture medium, which may contain serum or may be serum-free. In general, a substrate which allows adherence of cells thereto may be any substantially hydrophilic substrate. Current standard practice for growing adherent cells may involve the use of defined chemical media with or without addition of bovine, human or other animal serum. In a particularly preferred embodiment, such culturing medium comprises serum. These media, which can be supplemented with appropriate mixture of organic or inorganic compounds may, besides providing nutrients and/or growth promoters, also promote the growth/adherence or the elimination/detachment of specific cell types. The added serum, besides providing nutrients and/or growth promoters, may also promote cell adhesion by coating the treated plastic surfaces with a layer of matrix to which cells can better adhere. As appreciated by those skilled in the art, the cells may be counted in order to facilitate subsequent plating of the cells at a desired density. In another particularly preferred embodiment, such culturing medium is serum-free.

A method for producing a cell-free conditioned medium as taught herein may comprise the step of obtaining human liver progenitor or stem cells by any of the above methods, culturing human liver progenitor or stem cells in a cell culture medium, and separating the cell culture medium from human liver progenitor or stem cells.

The environment in which the cells are plated may comprise at least a cell medium, in the methods of the invention typically a liquid medium, which supports the survival and/or growth of the isolated liver progenitor or stem cells. The liquid culture medium may be added to the system before, together with or after the introduction of the cells thereto. Preferred media formulations have been described above.

Said method can be performed by using the cell culture medium that is a serum-free medium, by modifying specific conditions of cell culture, and/or by separating the cell culture medium from said human liver progenitor or stem cells after culturing human liver progenitor or stem cells at given time points (e.g. at least 2, 4, 6, 8, 12, 24 hours). Obtained conditioned medium have a composition enriched (or depleted) in soluble proteins, RNAs, exosomes and/or microvesicles that are either degraded (or unstable) within the conditioned media or secreted by human liver progenitor or stem cells not in regular manner but only before or after a certain number of hours (and thus not progressively accumulated in the conditioned medium). Relevant time points can be shorter (e.g. 2 hours or less) or longer such as at 24 hours, at 36 hours or more hours. By obtaining samples of said conditioned medium at these time points or at intermediate ones (such as 1, 2, 4, 6, 8, 12, or 18 hours) and testing such samples for their composition and/or activities, the optimal timing for obtaining the desired conditioned medium derived from human liver progenitor or stem cells can be determined.

In one embodiment, said conditioned medium is a product derived from cell-free conditioned medium obtainable by culturing liver progenitor or stem cells. Said product is a fraction obtained by fractioning said conditioned medium. Such fractioning may comprise applying one or more technologies known in the art, such as for example filtering, enzymatically digesting, centrifuging, adsorbing, and/or separating by chromatography.

The conditioned medium of the invention and/or the fraction thereof, typically contain soluble proteins, RNAs, exosomes and/or microvesicles.

In a preferred embodiment, the conditioned medium comprises one or more components chosen from the group of hepatocyte growth factor (HGF), interleukin 6 (IL-6), interleukin 8 (IL-8) and vascular endothelial growth factor (VEGF).

In a further preferred embodiment, the conditioned medium and/or the fraction thereof comprises at least hepatocyte growth factor (HGF), interleukin 6 (IL-6), interleukin 8 (IL-8) and vascular endothelial growth factor (VEGF).

In certain embodiments the conditioned medium and/or the fraction thereof comprise:
(a) at least one of soluble proteins selected from the group consisting of: hepatocyte growth factor (HGF), vascular endothelial growth factor (VEGF), eotaxin (CCL11), interleukin-6 (IL-6), and interleukin-8 (IL-8); and, optionally
(b) at least one of soluble proteins selected from the group consisting of matrix metalloproteinases, growth factors, chemokines, and cytokines.

Such soluble proteins may be preferably present in the conditioned medium and/or in a fraction thereof, at a concentration of at least 1 ng/ml. In particular, one or more of HGF, VEGF, CCL11, IL-6, or IL-8 (preferably all of them) may be present at a concentration of at least 1 ng/ml.

In an alternative embodiment one or more soluble proteins selected from the group consisting of HGF, VEGF, CCL11, IL-6, and IL-8 are present in the conditioned medium and/or fraction thereof, at a concentration of at least 1 ng/ml/million cells.

In another embodiment the conditioned medium and/or the fraction thereof contain microvesicles that are characterized and, when appropriate, selected according to their size (in certain embodiments, size smaller than 0.40 µm), molecular weight, and/or composition.

In another and further embodiment the conditioned medium and/or the fraction thereof contain exosomes that are characterized (in certain embodiments, size smaller than 80 nm) and, when appropriate, selected according to their size, molecular weight, and/or composition.

In another preferred embodiment, the conditioned medium and the fraction thereof comprise RNA, for example miRNA.

Particularly desired concentrations of such soluble proteins, RNA, exosomes and/or microvesicles conditioned medium and/or in a fraction thereof, can be obtained for example by appropriately concentrating (or diluting) the respective preparation at least about 5-fold, at least about 10-fold, at least about 20-fold, at least about 50-fold, or at least about 100-fold. Hence, certain embodiments provide so-concentrated or so-diluted conditioned medium and/or in a fraction thereof

In a further preferred embodiment, the present invention provides a fraction obtainable from said conditioned medium, said fraction comprising hepatocyte growth factor (HGF), vascular endothelial growth factor (VEGF), eotaxin (CCL11), interleukin-6 (IL-6), and interleukin-8 (IL-8), each one being present at a concentration of at least 1 ng/ml.

In an alternative embodiment, the fraction obtained from said conditioned medium, comprises one or more soluble proteins selected from the group consisting of HGF, VEGF, CCL11, IL-6, and IL-8, each one, at a concentration of at least 1 ng/ml/million cells.

As mentioned, the conditioned medium and/or fraction thereof, is suitable for the use as described above. Such medical, e.g., prophylactic or therapeutic, use may involve using conditioned medium and/or fraction thereof, alone or in combination with one or more exogenous active ingredients, which may be suitably added. Examples of such exogenous active ingredients include cells (e.g., liver progenitor or stem cells or other cells suitable for ex vivo or in vivo applications), proteins (e.g., matrix metalloproteases, growth factors, chemokines, cytokines, hormones, antigens, or antibodies), nutrients (e.g., sugars or vitamins) and/or chemicals (e.g., drugs with antimicrobial, anti-inflammatory, or antiviral properties) that were not initially present in conditioned medium and/or fraction thereof, and that are known to be effective as medicaments for the desired indication.

In an embodiment of the invention, a cell lysate of the cells as described above is used, rather than the actual cells. Said cell lysate may be obtained by any means known in the art, such as enzyme digestion of cells or a cell culture, detergent and/or buffer treatment, and the like.

In a further preferred embodiment, the present invention provides pharmaceutical formulations comprising a pharmaceutically effective amount of liver progenitor or stem cells, lysates thereof and/or conditioned cell medium. The pharmaceutical formulations may optionally also further comprise a pharmaceutically effective amount of one or more exogenous active ingredients, which may be of the type discussed above, e.g., the cells, proteins, nutrients, and/or chemicals. The exogenous active ingredients may be of the type discussed above, e.g., the cells, proteins, nutrients, and/or chemicals.

Some mesenchymal progenitor and stem cells are known to attenuate the dysfunction of organs and improve survival in several models of sepsis in animals, suggesting its potential use for treatment of patients with sepsis mesenchymal stem cells have an intrinsic capacity to migrate to injured tissues, such as the lung, myocardium, brain, liver, and kidney, and can improve the lesion by reducing both local and systemic inflammation via a decrease in the production of pro-inflammatory cytokines and an increase in production of anti-inflammatory cytokines.

It has been unexpectedly found that liver progenitor or stem cells, cell lysate, and/or the conditioned medium obtainable by culturing of progenitor or stem cells in medium are effective in use in the treatment of diseases and/or conditions caused by increased vascular permeability or for use in restoring the vascular integrity of cells and tissues in a subject following inflammation and/or infection in said subject. Defective endothelial barrier function is a common feature of many disorders. The liver progenitor or stem cells, cell lysate or conditioned medium obtained by culturing said cells in medium can be used in treatment of diseases such as peripheral vascular diseases, stroke, heart disease, diabetes, insulin resistance, chronic kidney failure, tumor growth, metastasis, venous thrombosis and severe viral infection diseases.

In a further preferred embodiment, the present invention provides liver progenitor or stem cells, the cell lysate thereof and/or the conditioned medium obtainable by culturing of progenitor or stem cells in medium for use in the treatment (as a prophylaxis or a therapy) of a series of disorders and/or conditions, including but not limited to:
Pulmonary diseases (excluding adult respiratory distress syndrome) such as asthma, acute lung injury, ventilator-induced lung injury, and other conditions which lead to pulmonary edema;
Ischemic diseases, such as ischemia reperfusion injury and any other disorder which refers to a tissue damage after the return of a blood supply to previous ischemic areas, typically observed after myocardial infarction and organ transplantation;
Diabetes and ocular (retinal) diseases, as well as a series of complications that affect multiple organs, manifested as retinopathy, cardiomyopathy, nephropathy, cerebral and peripheral vascular diseases;
Diseases and disorders related to abnormal microcirculatory function and endothelial barrier injury;
Cancer, especially solid tumors, like sarcomas, carcinomas and endothelial dysfunction; and cancer related microcirculation disturbances;
Heart diseases, such as myocardial pathology, myocardial infarction, myocardial edema and ischemic heart diseases;
Clarkson’s disease; and
Sepsis and sepsis-induced disease, such as sepsis-induced myocardial edema, sepsis-induced kidney failure and lung sepsis.

The present cells, lysate thereof and/or conditioned medium can therefore be used in the treatment of vascular hyperpermeability related diseases and/or conditions including but not limited to heart diseases, pulmonary diseases, ischemic diseases, diabetes, ocular diseases, stroke, cancer, Clarkson’s disease and sepsis. It has been shown that most of the pro-inflammatory cytokines including IFN-γ, TNF-α and IL-1ß cause an increase in tight junctions permeability of the endothelial cells, while some of the anti-inflammatory cytokines such as IL-10 and TGF-ß protect against the disruption of intestinal tight junctions barrier and development of inflammation.

The liver progenitor or stem cells, cell lysate and/or conditioned medium obtained by culturing said cells in medium are suitable for the treatment of Clarkson’s disease in a subject.

The liver progenitor or stem cells, cell lysate and/or conditioned medium obtained by culturing said cells in medium are particularly suitable for treatment of vascular hyperpermeability triggered by an inflammation and/or infection in a subject.

In a preferred embodiment, said liver progenitor or stem cells, cell lysate and/or conditioned medium obtained by culturing said cells in medium are particularly suitable for treatment of infection induced increased vascular permeability, either Gram-positive (Streptococcus pneumoniae, Staphylococcus aureus, Bacillus cereus) or Gram-negative bacteria (Pseudomonas aeruginosa) induced increased vascular permeability, preferably Gram-negative bacteria induced increased vascular permeability, most preferably lipopolysaccharide induced increased vascular permeability.

As described above, due to the vascular integrity restoring properties of the liver progenitor or stem cells, cell lysate thereof, and/or conditioned medium obtained by culturing said cells in said medium can be administered to a subject, e.g. a human patient to protect tissues, organs and/or its organ system affected by an increased vascular permeability.

This is especially advantageous during microbial infection of a subject, particularly bacterial infection of the subject. In a particularly preferred embodiment, the liver progenitor or stem cells, cell lysate thereof, and/or conditioned medium obtained by culturing said cells in said medium is particularly suitable in treatment of sepsis or sepsis-induced disease in a subject. In particular, the sepsis-induced disease in a subject may be sepsis-induced myocardial edema, sepsis-induced acute kidney injury, or lung sepsis.

Sepsis is caused by an infection and involves a complex interaction between the pathogen and the host immune cells. Such state is characterized by a systemic inflammatory state. The role of immune response is crucial to fight infection; but it is also responsible for the inflammatory tissue infiltration and severe organ damage, both hallmarks of sepsis. Evidence suggests that modulation of pro- and anti-inflammatory factors contributes to the suppression of immune effector cells, induces the systemic inflammation and causes tissue damage during the sepsis. The liver progenitor or stem cells, cell lysate thereof, and/or conditioned medium obtained by culturing said cells in said medium are unexpectedly found to be potent modulators of immune responses, with the ability to regulate both the innate and adaptive immune response.

It has been found that the liver progenitor or stem cells, cell lysate thereof, and/or conditioned medium obtained by culturing said cells in said medium exert the protective function on endothelial integrity through the secretion of anti-permeability factors that in cascade, among others, activate AMP-activated protein kinase signaling.

In a preferred embodiment, said anti-permeability factors exerting the beneficial effect on the functional integrity on the vascular endothelium are at least factors selected from the group comprising of fibroblast growth factor (FGF) family, angiopoietin-1, sphingosine-1-phosphate (S1P), TGF-ß, PDGF, HGF, TIMP1, and TIMP2.

In an embodiment, the conditioned medium of current invention comprises at least one component from the group of FGF family, angiopoietin-1, sphingosine-1-phosphate, TGF-β, HGF, TIMP1, and TIMP2.

In another embodiment, the conditioned medium of current invention comprises at least one component selected from sphingosine-1-phosphate, TGF-β1, HGF and TIMP2.

In a further preferred embodiment, the conditioned medium of the current invention comprises at least sphingosine-1-phosphate (S1P).

The present invention also relates to a conditioned medium obtained from culturing human liver progenitor cells, comprising at least 30 ng/million total cells of sphingosine-1-phospate, preferably at least 50 ng/million total cells, at least 100 ng/million total cells, at least 150 ng/million total cells, at least 200 ng/million total cells, at least 250 ng/million total cells or at least 300 ng/million total cells. In an embodiment, the conditioned medium is obtained as described in any of the embodiments above or below.

Sepsis commonly leads to a state of immunosuppression characterized by lymphocyte apoptosis and susceptibility to nosocomial infections. However, clinicians also know that progressive subcutaneous and body-cavity edema typically develops in patients with sepsis, suggesting widespread increases in vascular permeability. The accumulation of parenchymal and interstitial fluid leads to a severe tissue edema which could impair organ function by increasing the distance required for the diffusion of oxygen and by compromising microvascular perfusion because of increased interstitial pressure. Cytokines and other inflammatory mediators induce gaps between endothelial calls by disassembly of intercellular junction, by altering the cellular cytoskeletal structure or by directly damaging the cell monolayer. The recovery from sepsis is manifested as spontaneous diuresis with reduction in edema, consistent with restoration of vascular integrity.

It has been unexpectedly found that the liver progenitor or stem cells, cell lysate thereof, and/or conditioned medium obtained by culturing said cells in said medium can promote the assembly of 4 types of cell junctions present in the walls of endothelial cells. Said cell junctions differ both in structure and function and are 1) tight junctions, 2) adherens junctions, 3) GAP junctions, and 4) syndesmos. Thus, the cells, lysate thereof, and/or conditioned medium of the invention are particularly effective in treatment of vascular permeability and restoration of normal vascular permeability which is the consequence of disturbance and malfunction of any of the four types of the cell junctions.

In another embodiment, the liver progenitor or stem cells, lysate thereof or conditioned medium obtained by culturing said cells in said medium exert antibacterial activity by reducing the number of bacterial colony forming units (CFU) present in septicemia state blood, spleen, peritoneal fluid and the like.

In another embodiment, the liver progenitor or stem cells, lysate thereof, and/or conditioned medium obtained by culturing said cells in medium can be used in the treatment of tumor-related abnormal vasculature, particularly solid tumor-related abnormal vasculature. Solid tumors can be conceptualized as “organs” in themselves, composed of cancer cells, stromal cells, immune cells, and blood and lymphatic vessels, all embedded in a matrix. It has been recognized for decades that most tumors are highly vascular. On the other hand, solid tumors are known to release the factors that promote the growth of disorganized, leaky blood vessel networks, that display disturbed blood flow, inflammatory cells infiltration and tumor cells extravasation.

The liver progenitor or stem cells and/or lysate thereof, and/or conditioned medium obtained by culturing said cells of the invention, are particularly suitable for sepsis and sepsis-induced diseases, where restoring of a physiological circulation and reducing edema are key factors for survival of a patient.

It should be understood that the liver progenitor or stem cells, lysate thereof, and/or conditioned medium obtained by culturing said cells in medium can be administered to a subject in need as such a mammal, more preferably a human, or alternatively, as a part of a suitable pharmaceutical composition, without departing from the scope of the invention. In one embodiment of the invention the liver progenitor or stem cells, cell lysate thereof, and/or conditioned medium are administered as a composition which further comprises a pharmaceutically acceptable carrier. The pharmaceutically acceptable carrier or diluent is chosen wherein the cells of the invention remain viable and retain their vascular repair and immunomodulatory properties. The carrier can be a pharmaceutically acceptable solvent or dispersing medium containing, for example, water, saline, phosphate buffered saline, polyol (for example, glycerol, propylene glycol, liquid polyethylene glycol, and the like) and suitable mixtures thereof. The invention thus discloses a pharmaceutical composition comprising the isolated liver progenitor or stem cells and/or cell lysate and/or conditioned medium. Preferably, a composition comprising the isolated liver progenitor cells of the invention may comprise at least 10³, 10⁶, 10⁹ or more cells (for example, between 5 million and 500 million or between 5 million and 250 million or between 50 million and 500 million or between 50 million and 250 million or between 100 million and 500 million or between 100 million and 250 million of cells for each dose or administration). Such cell-based compositions may also include other agents of biological (e.g. antibodies or growth factor) or chemical origin (e.g. drugs, cell preserving or labelling compounds) that may provide a further therapeutic, diagnostic, or any other useful effect. The literature provides several examples of optional additives, excipients, vehicles, and/or carrier that are compatible with cell-based pharmaceutical compositions that may include further specific buffers, growth factors, or adjuvants, wherein the amount of each component of the composition is defined (in terms of micrograms/milligrams, volume, or percentage), as well as the means to combine them with liver progenitor cells.

An issue concerning the therapeutic use of the progenitor or stem cells of the invention is the quantity of cells necessary to achieve an optimal effect. Doses for administration may be variable, may include an initial administration followed by subsequent administrations and can be ascertained by the skilled artisan armed with the present disclosure. Typically, the administered dose or doses will provide for a therapeutically effective amount of the cells, i.e., one achieving the desired local or systemic effect and performance. In addition, the skilled person can readily determine the optional additives, vehicles, and/or carrier in pharmaceutical compositions of the invention to be administered to a subject.

In a further embodiment the composition of the invention can be provided as pharmaceutical compositions that can be used in therapeutic methods for in vivo administration (in humans or in animal models) or in vitro applications in the form of a composition including isolated liver progenitor or stem cells, lysate thereof, and/or conditioned medium either as fresh or in formulation suitable for long-term storage (e.g. cryopreserved cells). These pharmaceutical compositions can be provided as a HALPC product, optionally combining HALPC with a liquid carrier (e.g. cell culture medium or buffer) that is appropriate for the desired method of treatment, the selected route of administration, and/or storage, as well as in the preferred means for providing such pharmaceutical compositions (e.g. within a kit). Other agents of biological (e.g. antibodies or growth factor) or chemical origin (e.g. drugs, preserving or labeling compounds) that may provide any other useful effect can be also combined in such compositions.

For example, the cells may be provided as a cell suspension in any preservation medium after isolation procedure or after thawing following cryopreservation. As an example, the cell suspension may be prepared with a sterile diluent, such as a sterile aqueous solution, optionally comprising excipients such as pH-modifiers and/or human serum albumin, which is physiologically compatible with the patient.

Sterile injectable solutions can be prepared by incorporating the cells utilized in practicing the present invention in the required amount of the appropriate solvent with various amounts of the other ingredients, as desired. Such compositions may further be in admixture with a suitable carrier, diluent, or excipient such as sterile water, physiological saline, glucose, dextrose, or the like. The compositions can contain auxiliary substances such as wetting or emulsifying agents, pH buffering agents, gelling or viscosity enhancing additives, preservatives, flavoring agents, colors, and the like, depending upon the route of administration and the preparation desired.

In another or further embodiment the composition of the invention can be provided as a suspension of cells, a sponge or other three-dimensional structure where cells can grow and differentiate in vitro and/or in vivo including bioartificial liver devices, natural or synthetic matrices, or other systems allowing the engraftment and functionality of cells in appropriate locations (including areas of inflammation or tissue injury that expressing chemokines that help the homing and the engraftment of the cells). In particular, the composition comprising liver progenitor or stem cells, lysates thereof, and/or conditioned medium of the invention can be administered via injection (encompassing also catheter administration, intravenously or intra-arterially) or implantation, e.g. localized injection, systemic injection, intrasplenic injection, intra-articular injection, intraperitoneal injection, intraportal injection, injection to liver pulp, e.g., beneath the liver capsule, parenteral administration, or intrauterine injection into an embryo or fetus.

The liver progenitor or stem cells, lysate thereof, and/or conditioned medium can be administered to a tissue of interest, preferably tissue and/or organ characterized by an increased vascular permeability, in a subject to support endothelial barrier and providing potential beneficial effect on the functional integrity of the vascular endothelium.

In any of the preceding embodiments, the composition comprising the liver progenitor cells or stem cells may be administered to the patient in the form of a sterile liquid.

Said sterile liquid may be prepared from a reconstituted suspension of the liver progenitor cells or stem cells, prepared for example by the dilution of a thawed concentrated the liver progenitor cells or stem cell suspension with a sterile diluent, such as a sterile aqueous solution, optionally comprising excipients such as pH-modifiers and/or human serum albumin.

The invention is further described by the following non-limiting examples which further illustrate the invention, and are not intended to, nor should they be interpreted to, limit the scope of the invention.

### EXAMPLES

### Example 1: Production of Cells and Conditioned Medium

Cells were obtained from three different donors according to the protocol described in WO 2007 071 339, WO 2016 030 525, or EP 3 423 566.

Human adult liver progenitor cells were isolated, as described in EP 3 140 393 or EP 3 423 566, from livers of healthy cadaveric or non-heart beating donors.

Briefly, liver cell preparations are re-suspended in Williams′ E medium supplemented with 9% FBS, 10 mg/ml INS, 1 mM DEX and 1% P/S. The primary cells are cultured on Corning® CellBIND® flasks and cultured at 37° C. in a fully humidified atmosphere containing 5 % CO₂. After 24 hours, medium is changed in order to eliminate the non-adherent cells and thereafter renewed twice a week, whereas the culture is microscopically followed every day. Culture medium is switched after 12-16 days to high glucose DMEM supplemented with 9% FBS with or without 0.9% P/S. A cell type with mesenchymal-like morphology emerges and proliferates. When reaching 70-95% confluence, cells are trypsinized with recombinant trypsin and 1 mM EDTA, and re-plated at a density of 1-10 × 10³ cells/cm². At each passage, cells were trypsinized at 80-90 % confluency.

The conditioned medium was obtained as described hereafter: at passage 4 or 5, the cells were washed and the culture medium was replaced by fresh culture medium (DMEM + 0.5 to 10% FBS). After 6 to 96 hours, generally 24 hours incubation, supernatants were collected and cleared from floating cells and debris by means of centrifugation and stored for further assays. The concentration of secreted protein is expressed as nanograms (ng) or picograms (pg) that are secreted in 24 hours by 10⁶ cells.

The presence of the following potential soluble mediators of endothelial permeability in the conditioned medium was assayed using ELISA: Angiopoietin-1, Sphingosine-1-phosphate, TGF-β1, HGF and TIMP2. Results are listed below, data are based on n = 2 batches.

| | | |
|---|---|---|
| Mediator | Secreted in cell medium? | Secretion level (indicative) |
| Angiopoietin-1 | Yes | 0 to 3500 pg/million total cells |
| Sphingosine-1-phosphate | Yes | 300 to 2500 ng/million total cells |
| ΤGF-β1 | Yes | 1000 to 2000 pg/million total cells |
| HGF | Yes | 2000 to 30000 pg/million total cells |
| TIMP2 | Yes | 50 - 200 ng/million total cells |

These ELISA results demonstrate a mid to high presence of at least sphingosine-1-phosphate, TGF-β1, HGF and TIMP2, secreted by the cells in the conditioned medium. In addition, the ELISA assay showed a variable and low presence of angiopoietin-1 secreted in the conditioned medium.

The effect of the conditioned media on the cell junctions and vascular permeability of human dermal blood epithelial cells (HDBECs) was checked. Three different types of analysis have been performed: immunostainings of inter-endothelial junction (IEJ) markers, permeability assay, AMPK phosphorylation as described in examples 2, 3, 4 and 5.

### Example 2: Morphology of Inter-Endothelial Junctions (IEJ), Immunostainings of IEJ Markers

For cell junction studies, HDBECs (2 × 10⁴ cells) were plated on coverslips placed in 24-well plates and grown to confluence in complete EGM-2MV medium (1 ml) containing 5% FBS at 37° C. in a humidified atmosphere with 5% CO₂. Alternatively, HDBECs (2 × 10⁴ cells) were exposed to the conditioned medium (CM, 500 µl) mixed with complete EGM-2MV medium and containing 10% FBS (500 µl) for 24 hours. In both cases, medium was changed to EGM-2MV supplemented with 1 % FBS for 2 hours prior to treatment with LPS (50 µg/ml) for 6 hours. After LPS stimulation, the HDBECs were fixed, permeabilized and immunostained with specific antibodies (ZO-1, Connexin 43, and VE-cadherin). 5-Aminoimidazole-4-carboxamide ribonucleotide (AICAR) is a potent pharmacological activator of AMPK that was used as positive control.

Slices were examined under fluorescence with a Zeiss Axio Imager microscope (Zeiss, Wetzlar, Germany). These analyses were run in biological triplicates (n=6 for each individual experiment).

### ZO-1 Immunostaining (400x)

Cell junctions-integrity was measured by an assessment of ZO-1 immunostaining. Impact of conditioned medium of the invention on ZO-1 organization was investigated by analysis of parameters such as density, regularity, linear pattern, and gap formation of the cells.

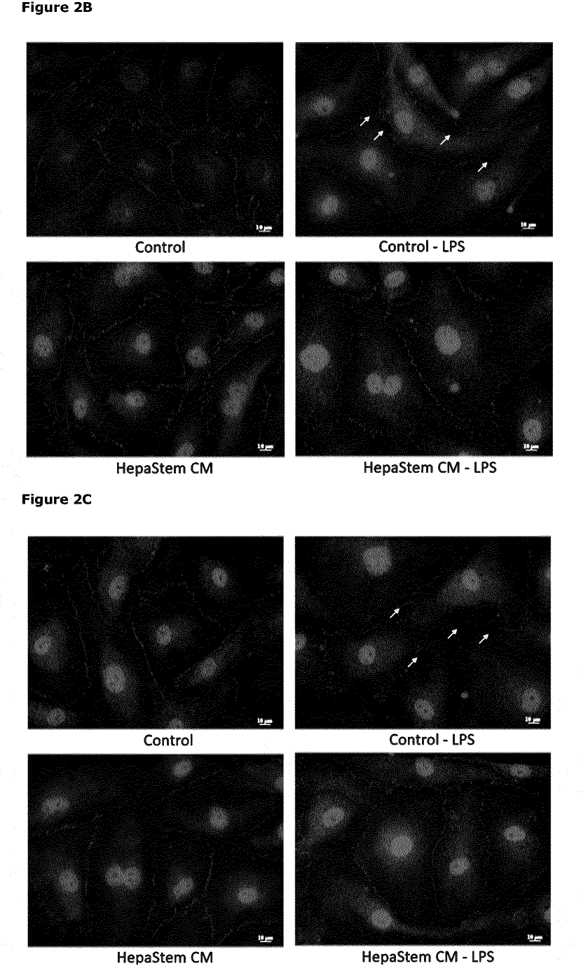

A comparison of HDBECs in basal state (Control) and of HDBECs submitted to LPS treatment (Control - LPS) confirms that LPS induces, as expected, irregularities in the linear expression pattern of ZO-1 at cell membrane. These irregularities seemed to be reduced when cells were treated with conditioned media of the invention (HepaStem CM - LPS). This has been observed in all donors tested (FIGS. 2A, 2B and 2C, for donors 1, 2 and 3, respectively). The images of HDBECs after treatment with conditioned medium without LPS stimulation (HepaStem CM) and with LPS stimulation (HepaStem CM - LPS) show few differences and confirm the protective role of conditioned medium of the invention in this test.

### Cx 43 Immunostaining (200x)

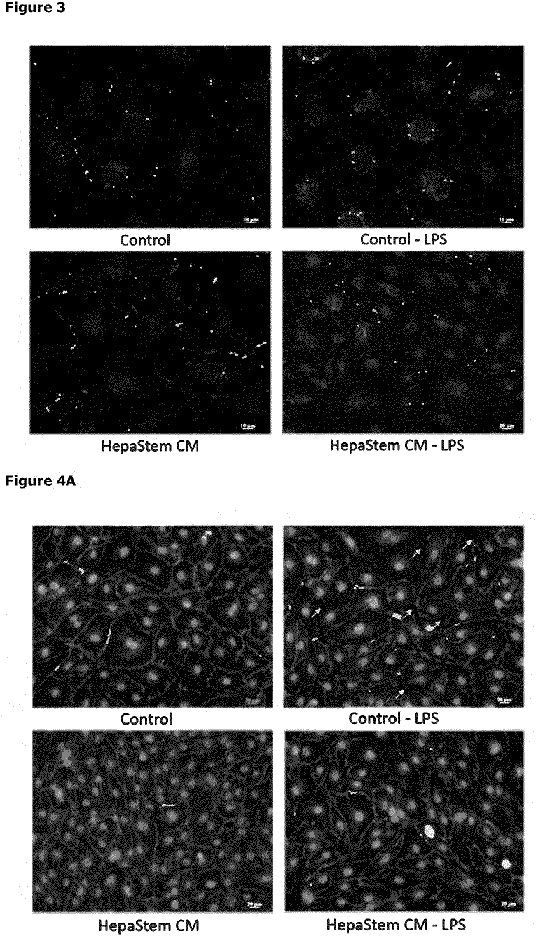

The effect of the conditioned medium of current invention on Gap junctions was evaluated by Connexin 43 (Cx43) immunostaining, in control group (control) and group treated with conditioned medium (HepaStem CM) with and without LPS treatment. The images of cells after treatment with conditioned medium and control groups are shown in FIG. 3 for donor 3. Contrarily to ZO-1, the staining of Cx43 did not show significant modulation induced by conditioned medium obtained by culturing liver progenitor or stem cells of the invention.

### VE-Cad Immunostaining (200x)

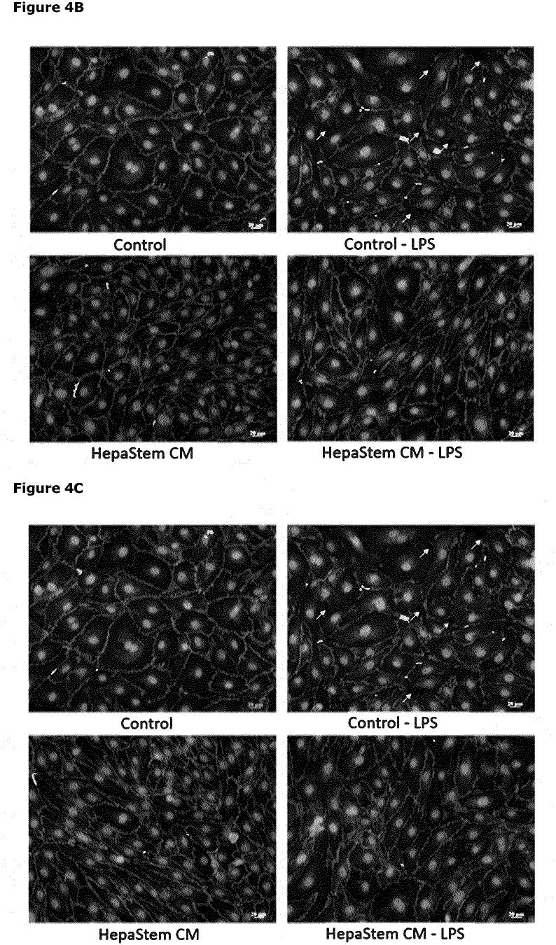

Adherens junctions of endothelial cells were evaluated by the immunostaining of VE-cadherin. The images are shown in FIG. 4 (200x) and highlight the reinforcement of the intercellular junctions by the conditioned medium in response to LPS challenge in comparison to the control. Said staining greatly confirmed the protective effect of the conditioned medium obtained by culturing of liver progenitor or stem cells of the invention on the preservation of the junctions’ integrity, similarly to the effect observed with ZO-1 for tight junctions.

### Example 3: Analysis of the Permeability of Endothelial Monolayers

The endothelial permeability assay was performed by using the Evans Blue Dye (EBD).

HDBECs (5 × 10⁴ cells) coated on Transwell inserts were cultured in complete EGM-2 MV medium (1 ml) containing 5% FBS or exposed to conditioned medium of the invention (CM, 500 µl) mixed with complete EGM-2MV medium containing 10% FBS (500 µl) for 24 hours. Medium was changed to EGM-2 MV supplemented with 1% FBS for 2 hours prior to Evans Blue Dye (EBD) loading. Immediately after, cells were treated with LPS (50 µg/ml) for 6 hours.

These analyses were run in biological triplicates (n=6 for each individual experiment).

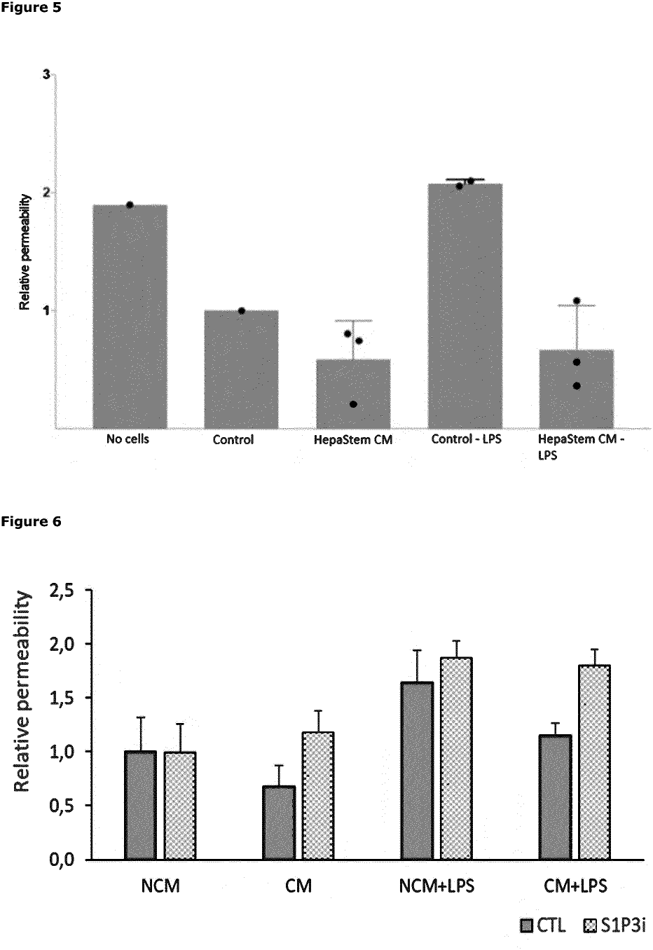

The effect of the conditioned medium of the invention on endothelial permeability assay is shown in FIG. 5. This assay showed similar cell permeability between non-conditioned medium NCM (GIBCO only media, no cells) and CM condition (the conditioned media of the invention) in absence of LPS challenge, whereas an important decrease in cell permeability was observed with conditioned media compared to NCM under LPS challenge. Observed effects suggested a protection of the inter-endothelial structure of the junctions upon addition of the conditioned media of the invention on dermal endothelial cells pre-treated with LPS. As the cells are first incubated 24 h with a mix (1:1) of HepaStem conditioned medium and endothelial cell medium, and secondly incubated for 1 h with low FBS followed by 6 h incubation with LPS, there is no more CM during the last 7 h of incubation. Therefore, the observed effect on endothelial permeability is preventive/protective and not restoration.

### Example 4: Permeability of Endothelial Monolayers Upon Blocking S1P Activity

The effect of HepaStem conditioned medium on LPS-induced endothelial cell permeability was tested in vitro using a Transwell assay using HBDECs and Evan Blue dye to assess permeability as described before (Example 3). A loss of function experiment was conducted in which a sphingosine-1-phosphate receptor inhibitor (S1P3 inhibitor) was used to block S1P activity (n=3 HepaStem batches). S1P is a compound highly secreted by the cells in the conditioned cell medium, as was shown in Example 1.

The relative endothelial cell permeability was tested in the following four conditions: non-conditioned medium (NCM) in the presence or absence of LPS challenge, or HepaStem conditioned medium (CM) in the presence or absence of LPS challenge. Each of these four conditions was subsequently treated with S1P3 inhibitor or mock (CM).

The results show that addition of S1P3 inhibitor in the presence of HepaStem CM increases endothelial permeability, indicating the involvement of S1P in the prevention of increased LPS-induced permeability. Results are shown in FIG. 6.

### Example 5: Assessment of AMPK Activation by Conditioned Medium of the Invention

HDBECs were grown in 6-well plates (10⁵ cells/plate) and exposed to the EGM-2MV medium (2 ml) containing 5 % FBS or exposed to the conditioned medium obtained with liver progenitor cells (CM, 1 ml) mixed with complete EGM-2MV medium containing 10% FBS (1 ml) for 24 hours. After treatment, cells were rinsed with PBS and lysed in SDS containing buffer. Protein extracts were separated on 4-20% gels and transferred to PVDF membrane. After staining with specific antibodies (anti-phospho-ACC, anti-ACC, anti-eiF2α) and membranes were developed using ECL.

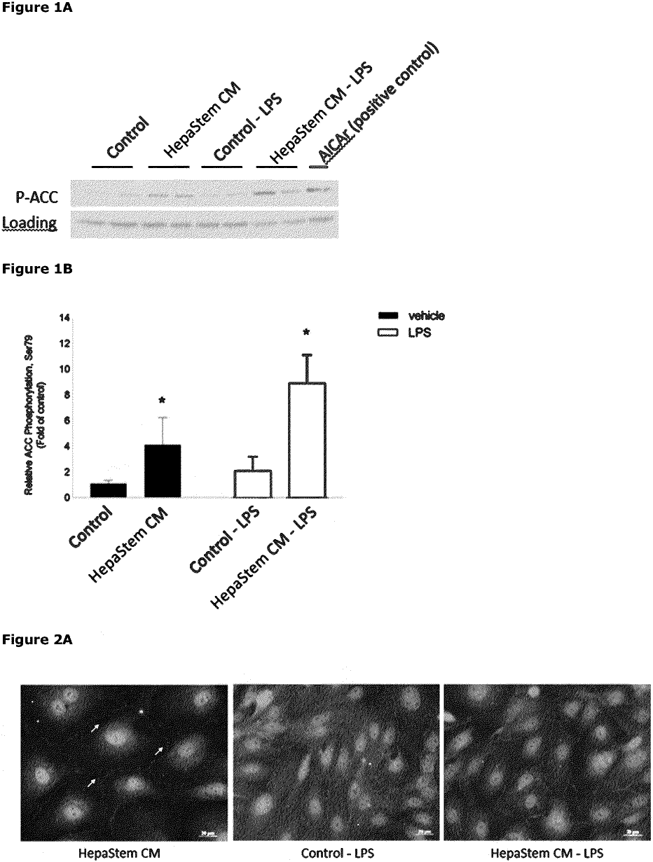

The referring to the average of the three donors tested (untreated liver progenitor or stem cells) are shown as FIG. 1. The acetyl-CoA carboxylase (ACC) is a downstream target of AMPK, whereas the 5-aminoimidazole-4-carboxamide ribonucleotide (AICAR) is a potent pharmacological activator of AMPK that was used as positive control.

The western blot analysis showed that conditioned media from liver progenitor or stem cells led to AMPK activation, as reflected by the significant increase of ACC phosphorylation. Notably, this increase reached higher levels in presence of LPS.

### Example 6: Effect of Liver Progenitor or Stem Cells on Myocardial Edema Induced by Endotoxemia

Septic cardiomyopathy is a well-recognized cardiovascular complication in patients with severe sepsis, characterized by a reversible decrease in systolic and/or diastolic left ventricular (LV) function, associated with left ventricular wall edema during sepsis. The in vivo model of endotoxemia in mice was used to evaluate the effect of liver progenitor or stem cells on myocardial edema. Briefly, experiments were performed on male C57BL/6 mice at age 8 weeks. The animals were maintained under a 12:12 hour light-dark cycle with free access to food and water. Endotoxemia was induced in mice by injecting intraperitoneally (IP) LPS (or saline vehicle). Evans blue dye (EBD), marker of albumin extravasation, was administrated IP (20 mg/kg) with LPS or saline vehicle, with or without prior administration of liver progenitor or stem cells or lysate thereof or conditioned medium. After 6 or 24 hours, the animals were euthanized with pentobarbital 300 mg/kg IP.

### Read-Out: Evaluation of the Vascular Leakage

The chest was open at 6 and 24 hours after LPS injection, and 30 mL of saline solution is flushed through the left ventricular (LV). The heart was removed and frozen. Vascular leakage, corresponding to the amount of dye in the extravascular compartment, was quantified by image J software as the relative surface of fluorescence (594 nm) on frozen sections (7 µm thickness).

### Read-Out: IHC Immunohistochemistry

Hearts were frozen. Seven µm heart sections were fixed with ice-cooled acetone or paraformaldehyde 4%. Tissues were then permeabilized in 0.1 % Triton X-100 and blocked with 5% BSA solution. Tissues were immunostained with anti-ZO-1 (1:50).

### Read-Out: Echocardiographic Assessment (Cardiac Functionality)

At baseline and 6 and 24 hours after LPS injection, mice were anesthetized by inhaled isoflurane, followed by echocardiography. Parasternal long- and short-axis views, four-chamber view, and mitral pulse wave Doppler were recorded. M-mode data were obtained from the parasternal long-axis view. LV dimensions were measured at end diastole and at end systole.

### Example 7: Effect of Liver Progenitor or Stem Cells in a Cecal Ligation and Puncture (CLP) Model

To study the etiology of human sepsis, researchers have developed different animal models. Polymicrobial sepsis induced by cecal ligation and puncture (CLP) is the most frequently used model because it closely resembles the progression and characteristics of human sepsis. The CLP model consists on the perforation of the cecum allowing the release of fecal material into the peritoneal cavity to generate an exacerbated immune response induced by polymicrobial infection. The polymicrobial sepsis is generated from fecal spillage after needle puncture.

The effect of liver progenitor or stem cells on polymicrobial sepsis was evaluated in C57BL/6 mice, preferably female mice at age 8 weeks. It has been observed that female mice are more resistant than males against sepsis-induced lethality, while mice older than 8 weeks produce less variable results than younger mice in terms of survival after CLP.

Approximately n=10 mice per group were used for survival analysis. Animals were weighted and anesthetized by injecting intraperitoneally (IP) ketamine (75 mg/kg) and xylazine (15 mg/kg). By using a scalpel, a small longitudinal skin incision was be made parallel and approximately 1 cm left to midline without penetrating in the peritoneal cavity. The cecum was ligated (60% ligation) to achieve a mid-grade sepsis. Note that the ligation equal to or more than 75% of the cecum generally results in high-grade sepsis, whereas the ligation equal to or less than 25% of the cecum results in low-grade sepsis. By using a 21 G needle, the cecum was perforated by one through-and-through puncture (two holes) near to the ligation. The direction of the perforation was from the mesenteric to the anti-mesenteric side of the cecum. Animals were then placed back in their cages with unlimited access to food and water. Mice were euthanized when they become moribund (indicated by clinical signs such as failure to move when touched or cyanosis). Mice were checked every hour to avoid having them die prior to euthanasia.

To assess the efficacy of cellular treatment, mice received an intravenously (IV) injection via tail vein of liver progenitor or stem cells of the invention at a dose ranging from 1.0 × 10⁵ to 1.0 × 10⁶ cells in 200 µl of phosphate buffered saline (PBS) together with EBD, 2 hours post-CLP. Eventually, additional tail vein injections of 2.5 × 10⁵ cells in 200 µl of PBS were given at 24 and 48 hours post-CLP depending from the liver progenitor or stem cells half-lives once administrated IV. As non-cellular control group in survival experiments, PBS was administered in a volume of 200 µl, at the same time points.

### Read Out: Evaluation Vascular Leakage and In Vivo Histology

Kidneys, livers, and spleens were collected and frozen to assess injury scoring by histology. Tissues were frozen. Seven µm sections were fixed with ice-cooled acetone or paraformaldehyde 4%. Tissues were then permeabilized in 0.1% Triton X-100 and blocked with 5% BSA solution. Tissues were immunostained with anti-ZO-1 (1:50). Vascular leakage, corresponding to the amount of dye in the extravascular compartment, was quantified by image J software as the relative surface of fluorescence (594 nm) on frozen sections (7-µm thick).

### Example 8: Effect of Liver Progenitor or Stem Cells in a Sepsis-Associated Acute Kidney Injury Model

The experiment was performed on male C57BL/6 mice previously subjected to cecal ligation and puncture (CLP). 24 hours after cecal ligation and puncture operation, the septic mice developed kidney injury. After 3 hours from sepsis induction, mice received liver progenitor or stem cells intravenously at a dose of 10⁶ cells (in combination with EBD).

### Read-Out

In vivo histology was performed on frozen sections of kidneys, livers, lungs and spleens to assess vascular leakage and injury scoring (ZO-1 immunostaining on kidneys).

### Example 9: Effect of Liver Progenitor or Stem Cells on Lung Sepsis

### Method 1

A model for generation of lung sepsis consisted on the introduction of a sterile gelatin capsule in the peritoneal cavity containing Escherichia coli suspension and non-sterile fecal content. Briefly, animals were weighed and then anesthetized with a mixture of ketamine (80 mg/kg) and xylazine (20 mg/kg) intraperitoneally. The abdomen of each animal was shaved and cleaned with povidone-iodine solution. A 1 cm midline abdominal incision was made to expose the linea alba. The peritoneum was opened by blunt dissection and sepsis was induced by introducing a sterile gelatin capsule in the peritoneal cavity containing another sterile capsule with the Escherichia coli (3 µL; Ref. ATCC 25922) suspension and non-sterile fecal content (20 mg). Animals were then divided into three groups:
i) sham (mice implanted with an empty capsule and received a retro-orbital injection of 100 µL PBS + EBD);
ii) sepsis (sepsis-induced and mice received a retro-orbital injection of 100 µL PBS + EBD); and
iii) sepsis + MSCs (sepsis-induced and mice are treated with 1 × 10⁶ liver progenitor or stem cells in a retro-orbital injection of 100 µL PBS + EBD).

### Read-Out: Evaluation Vascular Leakage and In Vivo Histology

Lungs are collected and frozen to assess injury scoring and vascular leakage by histology (anti-ZO-1 staining, quantification of dye in the extravascular compartment).

### Method 2

Another model that could be used to induce lung sepsis consists of injecting lipopolysaccharide (LPS) from E. coli (12.5 mg/kg) intraperitoneally.

The animals will be then divided into the same groups as in the previous model.

It is supposed that the present invention is not restricted to any form of realization described previously and that some modifications can be added to the presented example of fabrication without reappraisal of the appended claims. For example, the present invention has been described referring to liver progenitor or stem cells, but it is clear that the invention can be applied to specific human liver progenitor cell line for instance or to any lysate obtained thereof.

1. Liver progenitor or stem cells, lysates thereof, and/or conditioned medium obtainable by culturing said liver progenitor or stem cells in said medium, for use in modulating or influencing impaired vascular permeability in (cells of) a subject.

2. Liver progenitor or stem cells, lysates thereof, and/or conditioned medium obtainable by culturing said liver progenitor or stem cells in said medium, for use in the treatment of diseases and/or conditions caused by increased vascular permeability.

3. Liver progenitor or stem cells, lysates thereof, and/or conditioned medium obtainable by culturing said liver progenitor or stem cells in said medium, for use according to claim 1, wherein said cells are positive for at least one of markers selected from CD90, CD44, CD73, CD13, CD140b, CD29, vimentin and α-smooth muscle actin (ASMA), and optionally secrete HGF and/or PGE2.

4. Liver progenitor or stem cells, lysates thereof, and/or conditioned medium obtainable by culturing said liver progenitor or stem cells in said medium, for use according to claim 1, wherein said cells are positive for at least one of the markers chosen from the group of α-smooth muscle actin (ASMA), albumin (ALB), CD140b and MMP1, and negative for at least one of the markers chosen from the group sushi domain containing protein 2 (SUSD2) and cytokeratin-19 (CK-19).

5. Liver progenitor or stem cells, lysates thereof, and/or conditioned medium obtainable by culturing liver progenitor or stem cells in medium, for use according to claim 1, wherein said cells are measured:
- positive for α-smooth muscle actin (ASMA), CD140b and optionally albumin (ALB);
- negative for Cytokeratin-19 (CK-19) and optionally for Sushi domain containing protein 2 (SUSD2).

6. Liver progenitor or stem cells, lysates thereof, and/or conditioned medium obtainable by culturing liver progenitor or stem cells in medium, for use according to claim 1, wherein said cells are measured positive for CD90, CD73, vimentin and ASMA.

7. Liver progenitor or stem cells, lysates thereof, and/or conditioned medium obtainable by culturing liver progenitor or stem cells in medium, for use according to claim 1, wherein said cells are human.

8. Conditioned medium obtainable by culturing said liver progenitor or stem cells in said medium, for use according to claim 1, wherein said conditioned medium comprises one or more components from the group of hepatocyte growth factor (HGF), interleukin 6 (IL-6), interleukin 8 (IL-8) and vascular endothelial growth factor (VEGF).

9. Conditioned medium obtainable by culturing liver progenitor or stem cells in medium, for use according to claim 1, wherein said conditioned medium comprises at least one component from the group of fibroblast growth factor protein, angiopoietin-1, sphingosine-1-phospate, TGF-β, PDGF, HGF, TIMP1 and TIMP2.

10. Conditioned medium obtainable by culturing liver progenitor or stem cells in medium, for use according to claim 1, wherein said conditioned medium comprises at least one component from the group of sphingosine-1-phospate, TGF-β1, HGF and TIMP2.

11. Conditioned medium obtainable by culturing liver progenitor or stem cells in medium, for use according to claim 1, wherein said conditioned medium comprises at least sphingosine-1-phospate, preferably at a minimal concentration of 30 ng/million total cells.

12. Liver progenitor or stem cells, lysates thereof, and/or conditioned medium obtainable by culturing liver progenitor or stem cells in medium for use according to claim 1, wherein said diseases and/or conditions caused by increased vascular permeability are chosen from the group of heart, pulmonary and ischemic diseases, diabetes and ocular diseases, cancer (solid tumors), Clarkson’s disease and sepsis in a subject.

13. Liver progenitor or stem cells, lysates thereof, and/or conditioned medium obtainable by culturing liver progenitor or stem cells in medium for use according to claim 1, wherein said disease and/or condition caused by increased vascular permeability is triggered by an infection in a subject.

14. Liver progenitor or stem cells, lysates thereof, and/or conditioned medium obtainable by culturing liver progenitor or stem cells in medium for use according to claim 1, wherein said impaired vascular permeability is linked to, or said disease and/or condition is a sepsis or sepsis-induced disease in a subject.

15. Liver progenitor or stem cells, lysates thereof, and/or conditioned medium obtainable by culturing liver progenitor or stem cells in medium for use according to claim 1, wherein said impaired vascular permeability is linked to, or said disease and/or condition is a sepsis-induced myocardial edema in a subject.

16. Liver progenitor or stem cells, lysates thereof, and/or conditioned medium obtainable by culturing liver progenitor or stem cells in medium for use according to claim 1, wherein said impaired vascular permeability is linked to, or said disease and/or condition is a sepsis-induced acute kidney injury in a subject.

17. Liver progenitor or stem cells, lysates thereof, and/or conditioned medium obtainable by culturing liver progenitor or stem cells in medium for use according to claim 1, wherein said impaired vascular permeability is linked to, or said disease and/or condition is a lung sepsis in a subject.

18. Liver progenitor or stem cells, lysates thereof, and/or conditioned medium obtainable by culturing liver progenitor or stem cells in medium for use according to claim 1, wherein said impaired vascular permeability is linked to, or said disease and/or condition is Clarkson’s disease in a subject.

19. Liver progenitor or stem cells, lysates thereof, and/or conditioned medium obtainable by culturing liver progenitor or stem cells in medium for use according to claim 1, wherein the cells, lysates and/or medium are administered in a sterile liquid composition.

20. Conditioned medium obtained from culturing human liver progenitor cells, comprising at least 30 ng/million total cells of sphingosine-1-phospate.
